(19) United States
(12) Patent Application Publication
(10) Pub. No.: US 2020/0325178 A1
(43) Pub. Date: Oct. 15, 2020

(54) PEPTIDE NUCLEIC ACID MOLECULES FOR TREATMENT OF GRAM POSITIVE BACTERIAL INFECTION

(71) Applicant: Techulon Inc., Blacksburg, VA (US)

(72) Inventors: Nrusingh Prasad MOHAPATRA, Christiansburg, VA (US); Denis K. GUENNET, Blacksburg, VA (US); Bruhaspathy MIRIYALA, Wexford, PA (US)

(73) Assignee: Techulon Inc., Blacksburg, VA (US)

(21) Appl. No.: 16/760,647

(22) PCT Filed: Nov. 1, 2018
(86) PCT No.: PCT/US2018/058745

§ 371 Date: Apr. 30, 2020

Related U.S. Application Data
(60) Provisional application No. 62/580,093, filed on Nov. 1, 2017.

(51) Int. Cl. C07K 14/00; C12N 15/113; A61K 47/64; A61P 31/04

(52) U.S. Cl. CPC C07K 14/003; C12N 15/113; A61K 38/00; A61P 31/04; A61K 47/6455

(57) ABSTRACT

Disclosed are compositions for the treatment of Gram-positive bacteria infection and inhibition of Gram-positive bacteria growth. The compositions comprise a peptide nucleic acid linked to a cell-penetrating peptide (PNA-CPP). The PNA-CPP conjugate and compositions inhibit expression of bacterial proteins and are optionally administered in the form of nanoparticle compositions and antimicrobial fabrics.

### GOVERNMENT INTEREST

This work is based in part by the Defense Advanced Research Project Agency under Phase I SBIR contract number W911QX-12-C-0072. The US government has certain rights to the invention.

### REFERENCE TO SEQUENCE LISTING SUBMITTED ELECTRONICALLY

The content of the electronically submitted sequence listing in ASCII text file (Name: 3344.019PC01_SequenceListing.TXT; Size: 64,663 bytes; and Date of Creation: Oct. 30, 2018) filed with the application is incorporated herein by reference in its entirety.

### FIELD OF THE INVENTION

The field of the invention provides to peptide nucleic acids (PNAs) conjugated to a cell-penetrating peptide. The PNA-CPP conjugates targeting bacterial proteins are useful for treatment and inhibition of Gram positive bacterial infection.

### SUMMARY OF THE INVENTION

Provided are peptide nucleic acid (PNA) molecules conjugated to a cell-penetrating peptide (CPP). The PNA-CPP conjugates are useful for treatment of Gram positive bacterial infections and the inhibition of Gram positive bacterial growth. The PNA-CPP conjugates target bacterial membrane stability proteins and ribosomal proteins. In one embodiment, the PNA-CPP conjugate is complementary to a coding region of Staphylococcus aureus multimodular transpeptidase-transglycosylase/penicillin-binding protein 1A/1B (PBP1) protein. Embodiments of the PNA-CPP conjugate are shown in FIGS. 1-5. In one embodiment, the PNA-CPP conjugate is substantially pure. Also provided are pharmaceutical compositions comprising the PNA-CPP conjugates of the invention.

The invention also provides linkers for conjugating the CPP molecule to the PNA.

The invention also provides a method of inhibiting the growth of Gram positive bacteria, comprising administering the PNA-CPP conjugate or composition of the invention to a tissue containing said Gram positive bacteria or suspected of containing Gram positive bacteria. In one embodiment, the administering is topical administration. In another embodiment, the composition is in the form of a hygiene wipe. In another embodiment, the composition is in the form of an antimicrobial fabric.

The invention also provides a method of treating Gram positive bacterial infection, comprising administering to an animal in need thereof an effective amount of the PNA-CPP conjugate or composition of the invention.

### BRIEF DESCRIPTION OF THE DRAWINGS

FIG. 1 shows the structure of the PNA-CPP conjugate comprising an 8-amino-3,6-dioxaoctanoic acid (AEEA) linker (Compound I).

FIG. 2 shows the structure of the PNA-CPP conjugate comprising a 5-amino-3-oxapentanoic acid (AEA) linker (Compound II).

FIG. 3 shows the structure of the PNA-CPP conjugate comprising a glycine-glycine linker (Compound III).

FIG. 4 shows the structure of the PNA-CPP conjugate comprising an 8-amino-3,6-dioxaoctanoic acid (AEEA) linker, wherein arginine in the CPP is homo-arginine (Compound IV).

FIG. 5 shows the structure of the PNA-CPP conjugate comprising an 8-amino-3,6-dioxaoctanoic acid (AEEA) linker, wherein arginine in the CPP is D-arginine (Compound V).

FIG. 6 shows a growth curve of MRSA over 24 hours in presence of increasing amounts of Compound I-HCl determined in a Bioscreen-C spectrophotometer.

FIG. 7 shows a growth curve of MRSA over 24 hours in presence of increasing amounts of Compound II-HCl determined in a Bioscreen-C spectrophotometer

FIG. 8 shows a growth curve of MRSA over 24 hours in presence of increasing amounts of Compound III-HCl determined in a Bioscreen-C spectrophotometer.

FIG. 9 shows a growth curve of MRSA over 24 hours in presence of increasing amounts of Compound IV-HCl determined in a Bioscreen-C spectrophotometer.

FIG. 10 shows a growth curve of MRSA over 24 hours in presence of increasing amounts of Compound V-HCl determined in a Bioscreen-C spectrophotometer.

FIG. 11 shows the CFU counts for MRSA cultures incubated with increasing amounts of Compounds I-IV for 24 hours.

FIG. 12 shows a growth curve of MSSA over 24 hours in presence of increasing amounts of Compound I-HCl determined in a Bioscreen-C spectrophotometer.

FIG. 13 shows a growth curve of MSSA over 24 hours in presence of increasing amounts of Compound II-HCl determined in a Bioscreen-C spectrophotometer

FIG. 14 shows a growth curve of MSSA over 24 hours in presence of increasing amounts of Compound III-HCl determined in a Bioscreen-C spectrophotometer.

FIG. 15 shows a growth curve of MSSA over 24 hours in presence of increasing amounts of Compound IV-HCl determined in a Bioscreen-C spectrophotometer.

FIG. 16 shows a growth curve of MSSA over 24 hours in presence of increasing amounts of Compound V-HCl determined in a Bioscreen-C spectrophotometer.

FIG. 17 shows the CFU counts for MSSA cultures incubated with increasing amounts of Compounds I-IV for 24 hours.

FIG. 18 shows the MRSA CFU counts after exposure to either Compound I (6.25 μg/ml), Compound IV (6.25 μg/ml), or control for a period of eight days.

FIG. 19A-19B show the MRSA CFU counts for persister cells after exposure to Compound I (FIG. 19A) or Compound IV (FIG. 19B) for a period of eight days.

FIG. 20 shows the MSSA CFU counts after exposure to either Compound I (6.25 μg/ml), Compound IV (6.25 μg/ml), or control for a period of eight days.

FIG. 21A-21B show the MSSA CFU counts for persister cells after exposure to Compound I (FIG. 21A) or Compound IV (FIG. 21B) for a period of eight days.

FIG. 22 shows the MRSA CFU counts isolated from the thighs of animals infected with MRSA and treated with Compound I, vancomycin, or vehicle control.

### BRIEF DESCRIPTION OF THE SEQUENCE LISTING

The polynucleotide sequences in the sequence listing include the coding sequences for Staphylococcus aureus ribosomal proteins (SEQ ID NOs: 81-117) and membrane stability proteins (SEQ ID NOs: 118-131).

The polynucleotide sequences in the sequence listing also include antisense deoxyribonucleic acids (DNA) and/or modified nucleic acids, such as peptide nucleic acids (PNA). These sequences are capable of knockdown of expression of at least the following Staphylococcus aureus ribosomal and membrane stability proteins as set forth in Table 1:

TABLE 1
Antisense Polynucleotides Targeting Ribosomal
and Membrane Stability Proteins

| | Antisense | SEQ |
|---|---|---|
| | Polynucleotide | ID |
| Protein Target | Sequence | NO |
| LSU ribosomal protein L15p | tttcatttcggcacc | 1 |
| (L27Ae) | | |
| SSU ribosomal protein S17p | cgctcacttttgtaa | 2 |
| (S11e) | | |
| SSU ribosomal protein S7p | acgaggcataa | 3 |
| (S5e) | | |
| LSU ribosomal protein L28p | tgtttacccata | 4 |
| LSU ribosomal protein L27p | aacatcggaatg | 5 |
| LSU ribosomal protein L20p | actcgtggcata | 6 |
| SSU ribosomal protein S4p | cgagccataata | 7 |
| (S9e) | | |
| LSU ribosomal protein Ll3p | acgcataataat | 8 |
| (L13Ae) | | |
| SSU ribosomal protein S11p | ttacgtgccatt | 9 |
| (S14e) | | |
| SSU ribosomal protein S13p | tacgtgccatat | 10 |
| (S18e) | | |
| SSU ribosomal protein S5p | cgagccatgtat | 11 |
| (S2e) | | |
| LSU ribosomal protein L6p | tcatgttatggc | 12 |
| (L9e) | | |
| LSU ribosomal protein L14p | gttggatcatta | 13 |
| (L23e) | | |
| SSU ribosomal protein S17p | tctttcgctcac | 14 |
| (S11e) | | |
| SSU ribosomal protein S19p | tacgagccattt | 15 |
| (S15e) | | |
| LSU ribosomal protein L2p | tagccattgtcg | 16 |
| (L8e) | | |
| LSU ribosomal protein L3p | catcgaaagtcc | 17 |
| (L3e) | | |
| SSU ribosomal protein S6p | gttctcattttatat | 18 |
| LSU ribosomal protein L11p | tagccacgatgtgca | 19 |
| (L12e) | | |
| LSU ribosomal protein L1p | ttagccatttatagt | 20 |
| (L10Ae) | | |
| LSU ribosomal protein L10p | agacattcagacacc | 21 |
| (P0) | | |
| SSU ribosomal protein S12p | gttggcatgtgatat | 22 |
| (S23e) | | |
| SSU ribosomal protein S7p | tttacgaggcataat | 23 |
| (S5e) | | |
| LSU ribosomal protein L32p | tactgccatgatata | 24 |
| LSU ribosomal protein L19p | tgatttgtcattata | 25 |
| ribosomal protein L7Ae | tatactcattttggg | 26 |
| family protein | | |
| SSU ribosomal protein S15p | aaattgccataatca | 27 |
| (S13e) | | |
| SSU ribosomal protein S21p | tttagacatctgtat | 28 |
| LSU ribosomal protein L27p | taacatcggaatgca | 29 |
| Potential ribosomal protein | cagtaatcataataa | 30 |
| LSU ribosomal protein L21p | agcaaacatactttg | 31 |
| SSU ribosomal protein S4p | gagccataataagac | 32 |
| (S9e) | | |
| LSU ribosomal protein L13p | ttgacgcataataat | 33 |
| (L13Ae) | | |
| SSU ribosomal protein S11p | tttacgtgccattta | 34 |
| (S14e) | | |
| SSU ribosomal protein S13p | tacgtgccatattaa | 35 |
| (S18e) | | |
| LSU ribosomal protein L30p | tttagccataactag | 36 |
| (L7e) | | |
| SSU ribosomal protein S5p | cgagccatgtatttg | 37 |
| (S2e) | | |
| LSU ribosomal protein L18p | gatcatttcaatact | 38 |
| (L5e) | | |
| LSU ribosomal protein L6p | actcatgttatggca | 39 |
| (L9e) | | |
| SSU ribosomal protein S14p | tttagccacttaatt | 40 |
| (S29e) Zinc-dependent | | |
| LSU ribosomal protein L5p | cggttcaaagtggga | 41 |
| (L11e) | | |
| LSU ribosomal protein L14p | tggatcattagttaa | 42 |
| (L23e) | | |
| LSU ribosomal protein L16p | ggtagtaacattatt | 43 |
| (L10e) | | |
| SSU ribosomal protein S3p | ttgacccacagtatt | 44 |
| (S3e) | | |
| LSU ribosomal protein L22p | ttccattaggatgtc | 45 |
| (L17e) | | |
| SSU ribosomal protein S19p | gagccatttgggcgc | 46 |
| (S15e) | | |
| LSU ribosomal protein L2p | agccattgtcgctta | 47 |
| (L8e) | | |
| LSU ribosomal protein L23p | ttccattatccgagc | 48 |
| (L23Ae) | | |
| LSU ribosomal protein L3p | ggtcatcgaaagtcc | 49 |
| (L3e) | | |
| LSU ribosomal protein L34p | gttttaccatgcaaa | 50 |
| Multimodular transpeptidase- | cgtcatacgcggtcc | 51 |
| transglycosylase/ | | |
| penicillin-binding | | |
| protein 1A/1B(PBP1) | | |
| UDP-N-acetylglucosamine 1- | atccatcgtaaatcc | 52 |
| carboxyvinyltransferase | | |
| Cell division protein FtsI | cattactacgca | 53 |
| (Peptidoglycan synthetase) | | |
| UDP-N-acetylglucosamine-N- | tttcgtcattaa | 54 |
| acetylmuramyl- | | |
| (pentapeptide) | | |
| pyrophosphoryl- | | |
| undecaprenol N- | | |
| acetylglucosamine | | |
| transferase | | |
| Multimodular transpeptidase- | tcatacgcggtc | 55 |
| transglycosylase/ | | |
| Penicillin- | | |
| binding protein | | |
| 1A/IB (PBP1) | | |
| Alanine racemase | ccgacatattac | 56 |
| UDP-N-acetylglucosamine | catcgtaaatcc | 57 |
| 1-carboxyvinyltransferase | | |
| UDP-N- | tgcatccaaactgaa | 58 |
| acetylmuramoylalanyl-D- | | |
| glutamate-L-lysine ligase | | |
| Glutamate racemase | attcatattcggtca | 59 |
| Phospho-N-acetylmuramoyl- | acaaaaatcataact | 60 |
| pentapeptide-transferase | | |
| Undecaprenyl pyrophosphate | ttaaacatggtcttt | 61 |
| synthetase | | |
| tRNA-dependent lipid II- | tactcattttatcaa | 62 |
| Gly-glycine ligase @ | | |
| tRNA-dependent | | |
| lipid II-GlyGly-glycine | | |
| ligase @ FemA, factor | | |
| essential for methicillin | | |
| resisitance | | |
| UDP-N-acetylglucosamine-N- | gattttcgtcattaa | 63 |
| acetylmuramyl-(pentapeptide) | | |
| pyrophosphoryl-undecaprenol | | |
| N-acetylglucosamine | | |
| transferase | | |
| UDP-N-acetylmuramate- | agtgtgtcattatat | 64 |
| alanine ligase | | |
| Proposed amino acid ligase | gtctcatgtgtttcc | 65 |
| found clustered with an | | |
| amidotransferase | | |
| D-alanine-D-alanine ligase | tgtcatttcgttttc | 66 |

The peptide sequences in the sequence listing include peptides that target and/or localize nucleic acids to bacterial cells and promote bacterial membrane permeation. See Table 2:

TABLE 2
Cell Penetrating Peptides

| | | SEQ |
|---|---|---|
| | | ID |
| Peptide Name | Amino Acid Sequence | NO. |
| KFF peptide | KFFKFFKFFK | 67 |
| RFF peptide | RFFRFFRFFR | 68 |
| Magainin 2 | GIGKWLHSAKKFGKAFVGEIMNS | 69 |
| Transportin 10 | AGYLLGKINLKALAALAKKIL | 70 |
| cyclic d,1-alpha- | KKLWLW | 71 |
| peptide | | |
| cyclic d,1-alpha- | RRKWLWLW | 72 |
| peptide | | |
| cyclic d,1-alpha- | KQRWLWLW | 73 |
| peptide | | |
| amphipathic | LLIILRRRIRKQAHAHSK | 74 |
| peptide | | |
| PENETRATIN 1 | RQIKIWFQNRRMKWKK | 75 |
| peptide | | |
| TAT peptide | GRKKRRQRRRPQ | 76 |
| Indolicidin | ILPWKWPWWPWRR | 77 |

### Definitions

The term substantially pure means that the PNA-CPP conjugate is at least 95% homogeneous by HPLC. In another embodiment, the substantially pure PNA-CPP conjugate is 96% homogenous by HPLC. In another embodiment, the substantially pure PNA-CPP conjugate is 97% homogenous by HPLC. In another embodiment, the substantially pure PNA-CPP conjugate is 98% homogenous by HPLC. In another embodiment, the substantially pure PNA-CPP conjugate is 99% homogenous by HPLC. In another embodiment, the substantially pure PNA-CPP conjugate is 100% homogenous by HPLC.

### DETAILED DESCRIPTION OF THE INVENTION

The present invention may be understood by reference to the following detailed description of the embodiments of the invention and examples included herein. The terminology used herein is for the purpose of describing embodiments of the invention and is not intended to be limiting.

Specific aspects of the invention include a PNA-CPP conjugate that is useful for the treatment of Gram positive bacterial infection and/or inhibiting the growth of Gram positive bacteria. In some aspects, the PNA-CPP conjugate hybridizes to a coding region of Staphylococcal aureus multimodular transpeptidase-transglycosylase/penicillin-binding protein 1A/1B (PBP1) protein.

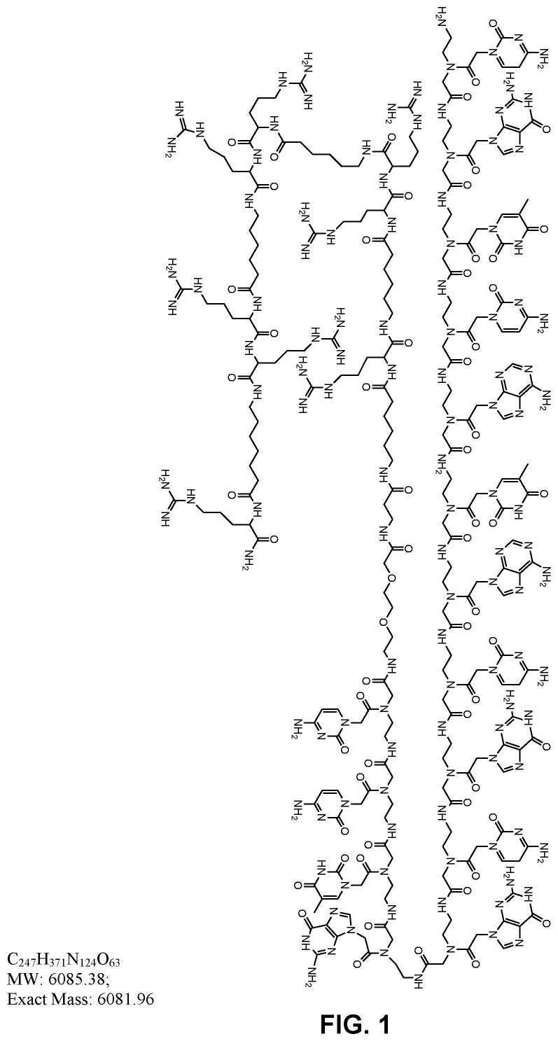

The PNA-CPP conjugates of the invention comprise a cell penetration peptide (CPP). The cell penetration peptide may have one or more functions to facilitate cell targeting and/or membrane permeation of Gram positive bacteria in a host. The cell penetration peptide provides for membrane disruption of bacteria provides specificity and reduces toxicity. Embodiments of the PNA-CPP conjugate utilizing different linkers are shown in FIGS. 1-5.

Bulk synthesis can be carried out by contract manufacturers, such as Neo Group, Inc. (Cambridge, Mass.) or AmbioPharm, Inc. (North Augusta, S.C.) using standard methodologies including solid-scaffold protection/deprotection synthesis via high fidelity synthesizers. In one embodiment, the PNA molecule is conjugated to the CPP using well known conjugation methods that employ succinimidyl-6-hydrazinonicotinateacetonehydrazone to succinimidyl-4-formylbenzoate coupling chemistry. This is a specific, well-behaved, and highly efficient conjugation method for peptide-DNA coupling. In order to covalently couple peptides to nucleic acids, the peptides are prepared for reaction by modifying the N-terminal with a reactive group. In one embodiment, the N-terminal of the peptide is modified with S6H (succinimidyl-6-hydrazinonicotinateacetonehydrazone). N-protected peptides are desalted and dissolved in dry DMF. Next, S6H is added in 2× molar excesses to a stirring solution and allowed to react at room temperature for 2 hours. Workup follows procedures known in the art, such as that described by Dirksen et al. J. Am. Chem. Soc. 2006 128, 15602-3. Other methods of coupling peptides to nucleic acids known in the art may be used.

In one embodiment of the invention, the PNA-CPP conjugate is part of a composition comprising a buffer. We found that the PNA-CPP conjugate exhibited greater antimicrobial activity in a composition comprising a basic pH. Thus, suitable buffers in the composition of the invention provide a basic pH when dissolved or dispersed in water. In some embodiments, the buffer has a pKa of greater than about 6. See, for example, “Handbook of Pharmaceutical Excipients,” 5^{th} ed., Rowe et al. (eds.) (2006); and SIGMA Life Sciences, “Products for Life Science Research,” Product Catalog (2008-2009). The composition may comprise one or more buffers. Such buffers include—but are not limited to—phosphate buffers, carbonate buffers, ethanolamine buffers, borate buffers, imidazole buffers, tris buffers, and zwitterionic buffers (e.g., HEPES, BES, PIPES, Tricine, and other so-called “Good's Buffers”). See, for example, Good et al., “Hydrogen Ion Buffers for Biological Research,” Biochemistry, 5(2):467-477 (1966). In one particular embodiment, the buffer is a carbonate, such as sodium bicarbonate or carbonate. In another particular embodiment, the buffer is imidazole. In another embodiment, the buffer is Tris(hydroxymethyl)aminomethane (“Tris”).

In one embodiment of the invention, the buffer has a pKa between about 6 and about 14, between about 7 and about 13, between about 8 and about 12, between about 9 and about 11, and between about 10 and about 11. In another embodiment, the buffer has a pKa between about 6 and about 9, between about 7 and about 9, and between about 8 and about 9. In another embodiment, the buffer has a pKa between about 6 and about 13, between about 6 and about 12, between about 6 and about 11, between about 6 and about 10, between about 6 and about 9, between about 6 and about 8, and between about 6 and about 7. In one embodiment the buffer has a pKa of 6.37. In another embodiment, the buffer has a pKa of 6.951n another embodiment, the buffer has a pKa of 8.1. In another embodiment, the buffer has a pKa of 10.25.

In another embodiment of the invention, the PNA-CPP conjugate is combined with a delivery polymer. The polymer-based nanoparticle drug delivery platform is adaptable to a diverse set of polynucleotide therapeutic modalities. In one aspect of the invention, the delivery polymer is cationic. In another aspect of the invention, the delivery polymer comprises phosphonium ions and/or ammonium ions. In another example of the invention, the PNA-CPP conjugate is combined with a delivery polymer, and the composition forms nanoparticles in solution. In a further embodiment, nanoparticle polyplexes are stable in serum and have a size in the range of about 30 nm-5000 nm in diameter. In one embodiment, the particles are less than about 300 nm in diameter. For example, the nanoparticles are less than about 150 nm in diameter.

In one embodiment, the delivery vehicle comprises a cationic block copolymer comprising phosphonium or ammonium ionic groups as described in PCT/US12/42974. In one embodiment, the polymer is diblock-Poly[(ethylene glycol)₉ methyl ethyl methacralate][stirylphosphonium]. In another embodiment of the invention, the delivery polymer comprises glycoamidoamines as described in Tranter et al. Amer Soc Gene Cell Ther, December 2011; polyhydroxylamidoamines, dendritic macromolecules, carbohydrate-containing polyesters, as described in US20090105115; and US20090124534. In other embodiments of the invention, the nucleic acid delivery vehicle comprises a cationic polypeptide or cationic lipid. An example of a cationic polypeptide is polylysine. See U.S. Pat. No. 5,521,291.

In one embodiment, the PNA-CPP conjugate is part of a composition comprising delivery or carrier polymers. In another embodiment, the PNA-CPP conjugate is part of nanoparticle polyplexes capable of transporting molecules with stability in serum. The polyplex compositions comprise a synthetic delivery polymer (carrier polymer) and biologically active compound associated with one another in the form of particles having an average diameter of less than about 500 nm, such as about 300 nm, or about 200 nm, preferably less than about 150 nm, such as less than about 100 nm. The invention encompasses particles in the range of about 40 nm-500 nm in diameter.

In one embodiment, the delivery or carrier polymer comprises a cationic block copolymer containing phosphonium or ammonium ionic groups as described in PCT/US12/42974. In another embodiment of the invention, the delivery or carrier polymer comprises glycoamidoamines as described in Tranter et al. Amer Soc Gene Cell Ther, December 2011; polyhydroxylamidoamines, dendritic macromolecules, carbohydrate-containing polyesters, as described in US20090105115; and US20090124534. The polyglycoamidoamine (PGAA) polymer system, which is a proprietary, localized and biodegradable nanoparticle system, represents another delivery or carrier polymer. Poly(galactaramidoamine) is an efficient cationic polymeric vehicle with low cytotoxicity (Wongrakpanich et al. Pharmaceutical Development and Technology, Jan. 12, 2012). The nanoparticle delivery system disclosed in Hemp et al. Biomacromolecules, 2012 13:2439-45 represents another delivery or carrier polymer useful in the present invention.

In other embodiments of the invention, the delivery or carrier polymer comprises a cationic polypeptide or cationic lipid. Polymers, such as poly-L-lysine (PLL), polyethyleneimine (PEI), chitosan, and their derivatives are also encompassed by the invention. Nucleic acid delivery using these compounds relies on complexation driven by electrostatic interactions between the gene and the polycationic delivery agent. Polymer-DNA complexes condense into particles on the order of 60 nm-120 nm in diameter. Polymers such as linear PEI and PLL have high transfection rates in a variety of cells.

In vivo nucleic acid delivery has size constraints requiring a sufficiently small polyplex to enable long circulation times and cellular uptake. In addition, polyplexes must resist salt- and serum-induced aggregation. Serum stability is generally associated with a particle size of about sub-150 nm hydrodynamic radius or below maintainable for 24 h. The nanoparticles of the invention, which comprise nucleic acid therapeutic and delivery polymer, have the hydrodynamic radius and material properties for serum stability. In particular, the delivery polymer, when combined with the nucleic acid, protects the therapeutic cargo under physiological conditions. The delivery polymers are designed to have characteristics of spontaneous self-assembly into nanoparticles when combined with polynucleotides in solution.

The invention also contemplates other delivery polymers that form serum-stable nanoparticles. The invention is not limited to the type of delivery polymer and may be adaptable to nucleic acid characteristics, such as length, composition, charge, and presence of coupled peptide. The delivery polymer may also be adaptable for material properties of the resultant nanoparticle, such as hydrodynamic radius, stability in the host bloodstream, toxicity to the host, and ability to release cargo inside a host cell.

In one embodiment, the PNA-CPP conjugate is administered in the form of a salt. The salt may be any pharmaceutically acceptable salt comprising an acid or base addition salt. Examples of pharmaceutically acceptable salts with acids include those formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997). Acid addition salts of basic molecules may be prepared by contacting the free base forms with a sufficient amount of the desired acid to produce the salt according to methods and techniques with which a skilled artisan is familiar.

Pharmaceutically acceptable base addition salts are formed by addition of an inorganic base or an organic base to the free acid. Pharmaceutically acceptable base addition salts may be formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like.

In one embodiment, the PNA-CPP conjugate is administered as part of a pharmaceutical composition comprising a pharmaceutically acceptable diluent, excipient or carrier. Suitable diluents, excipients and carriers are well known in the art and are described, for example, in Remington's Pharmaceutical Sciences, Mack Publishing Co. (A. R. Gernnaro Ed., 1985). The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, saline, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the PNA-CPP conjugate in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

In one embodiment, the composition comprising the PNA-CPP conjugate is in contact with a fabric. The fabric may comprise natural fibers, synthetic fibers, or both. Examples of textile fabrics include, but are not limited to, nylon, cotton, nylon-cotton blends, wool, silk, linen, polyester, rayon, and worsted. In one particular embodiment of the invention, the fabric is cotton. In another embodiment, the fabric is nylon. In another embodiment, the fabric is a nylon-cotton blend. The ratio of nylon to cotton in the nylon-cotton blend fabric can be between about 1:99 and about 99:1, between about 10:90 and about 90:10, between about 20:80 and about 80:20, between about 30:70 and about 70:30, between about 40:60 and about 60:40, and between about 45:55 and about 55:45. In a preferred embodiment, the fabric is a 50:50 nylon-cotton blend.

In another embodiment of the invention, the fabric has a high tensile strength-to-weight ratio. In one embodiment, the fabric with a high tensile-to-weight ratio is a fabric comprising aramid fibers. In a particular embodiment, the aramid fiber is a para-aramid fiber (e.g., the para-aramid fiber commercially known as KEVLAR). In another particular embodiment, the aramid fiber is a meta-aramid fiber (e.g., the meta-aramid fiber commercially known as NOMEX).

In certain embodiments, the antimicrobial fabric is capable of treating a Gram-positive bacterial infection or inhibiting growth of a Gram-positive bacteria after the fabric has been washed. In some embodiments, the antimicrobial fabric is capable of treating a Gram-positive bacterial infection or inhibiting growth of a Gram-positive bacteria after between about 10 and about 60 wash cycles, between about 20 and about 50 wash cycles, between about 20 and about 40 wash cycles, between about 20 and about 30 wash cycles, and between about 20 and about 25 wash cycles. In another embodiment, the duration of a wash cycle is between about 10 minutes and about 90 minutes, between about 10 minutes and about 75 minutes, between about 10 minutes and about 60 minutes, between about 10 minutes and about 45 minutes, between about 10 minutes and about 30 minutes, and between about 10 minutes and about 15 minutes. In another embodiment, the water temperature in the wash cycles is between about 16° C. and about 60° C., between about 27° C. and about 49° C., or between about 37° and about 44° C. In one particular embodiment, the antimicrobial fabric is capable of treating a Gram-positive bacterial infection or inhibiting growth of a Gram-positive bacteria following Laundry Test Method AATCC 147 from American Association of Textile Chemists and Colorists (AATCC).

In another embodiment, provided is a composition comprising the PNA-CPP conjugate. The composition may be in the form of solution that can be applied to a fabric, e.g., by rinsing, dipping, or spraying. The fabric can be an antimicrobial fabric or a non-antimicrobial fabric. In one embodiment, application of the solution to the fabric provides a fabric that is capable of treating a Gram-positive bacterial infection or inhibiting growth of a Gram-positive bacteria. In other embodiments, application of the solution to the fabric increases the fabric's capability of treating a Gram-positive bacterial infection or inhibiting growth of a Gram-positive bacteria. In a particular embodiment, application of the solution to an antimicrobial fabric with low antimicrobial activity increases the antimicrobial activity of the fabric.

In other embodiments of the invention, provide is a wound healing dressing comprising the PNA-CPP conjugate. In one embodiment, the wound healing dressing is an adhesive dressing. In another embodiment, the wound healing dressing is a non-adhesive dressing. In one embodiment, the dressing comprises a foam, gel, or cream. In another embodiment, the dressing comprises a fiber based material (e.g., gauzes or waddings). In one embodiment, the fiber-based material is cotton. In another embodiment, the fiber-based material is rayon. In another embodiment, the fiber-based material is a gel-forming fiber, such as a carboxymethylated cellulosic material. In another embodiment, the fiber-based material is a synthetic polymer. In another embodiment, the wound healing dressing is THERAGAUZE (Soluble Systems, LLC, Newport News, Va.).

The invention also provides a method of treating Gram positive bacterial infection and a method of inhibiting the growth of Gram positive bacteria. The Gram positive bacteria may include, but are not limited to, methicillin-resistant strains of Staphylococcus aureus (MRSA) and methicillin-susceptible strains of Staphylococcus aureus (MSSA). The Gram positive bacteria may also include, but are not limited to, other Staphylococcus spp. (e.g., vancomycin-resistant Staphylococcus aureus (“VRSA”) and S. epidermidis); Bacillus spp. (e.g., B. anthracis); Clostridium spp. (e.g., C. botulinum, C. dificile, C. perfringens, and C. tetani); Corynebacterium spp. (e.g., C. diptheriae); Enterococcus spp. (e.g., vancomycin-resistant Enterococcus spp. (“VRE”), E. faecalis, and E. faecium); Lysteria spp. (e.g., L. monocytogenes); Micrococcus spp. (e.g., M. luteus); Mycobacterium spp. (e.g., M. leprae and M. tuberculosis); Propionibacterium spp. (e.g., Propionibacterium acnes) and Streptococcus spp. (e.g., S. pneumoniae, S. pyogenes, and S. agalactiae). In one embodiment, the animal undergoing treatment for Gram positive bacterial infection exhibits one or more symptoms of Gram positive bacterial infection including puss production in the infected area, acne, boils, abscesses, carbuncles, stys, cellulitis, diarrhea, botulism, and gas gangrene. The animal may also exhibit signs of sepsis or pneumonia.

In one embodiment, the PNA-CPP conjugate is administered by intravenous injection. In another embodiment, the PNA-CPP conjugate is administered by intramuscular injection. In another embodiment, the PNA-CPP conjugate is administered by peritoneal injection. In another embodiment, the PNA-CPP conjugate is administered topically, e.g. to a tissue suspected to be infected by Gram positive bacteria. In another embodiment, the PNA-CPP conjugate is administered orally. When administered orally, the PNA-CPP conjugate may be formulated as part of a pharmaceutical composition coated with an enteric coating that will protect the PNA-CPP conjugate from the acid environment of the stomach and release the PNA-CPP conjugate in the upper gastrointestinal tract. In another embodiment, the PNA-CPP conjugate may be formulated as part of a sustained release formulation that will release the PNA-CPP conjugate on a substantially continuous basis over a period of time.

Animals that may be treated with the PNA-CPP conjugate according to the invention include any animal that may benefit from treatment with the PNA-CPP conjugate. Such animals include mammals such as humans, dogs, cats, cattle, horses, pigs, sheep, goats and the like.

The PNA-CPP conjugate is administered in an amount that is effective for the treatment of Gram positive bacterial infection or inhibition of the growth of Gram positive bacteria. The amount may vary widely depending on the mode of administration, the species of Gram positive bacteria, the age of the animal, the weight of the animal, and the surface area of the animal. The amount of PNA-CPP conjugate, salt and/or complex thereof may range anywhere from 1 pmol/kg to 1 mmol/kg. In another embodiment, the amount may range from 1 nmol/kg to 10 mmol/kg. When administered topically, the amount of PNA-CPP conjugate, salt and/or complex thereof may range anywhere from 1 to 99 weight percent. In another embodiment, the amount of PNA-CPP conjugate, salt and/or complex thereof may range anywhere from 1 to 10 weight percent.

The invention also provides PNA-CPP conjugates comprising a linker. In one embodiment, the PNA-CPP molecule is represented by the formula: N-L-Z, or pharmaceutically acceptable salt thereof, wherein N is an antisense molecule that inhibits the growth of a bacterium comprising a polynucleotide sequence that is antisense to the coding region of a bacterial protein and hybridizes to the coding region under physiological conditions; L is a linker having the formula (Y′)ₙ, where each Y′ is independently glycine, cysteine, 8-amino-3,6-dioxaoctanoic acid (AEEA), or 5-amino-3-oxapentanoic acid (AEA), and n is an integer from 1 to 10; and Z is a cell penetrating molecule. In some aspects, n is 1, 2, 3, 4, 5, 6, 7, 8, 9, or 10. In some aspects of the disclosure, N has a sequence selected from the group consisting of SEQ ID NOs: 1-66 (See Table 1).

In some aspects, the cell penetrating molecule Z has the formula: (ABC)ₚ-D, wherein A is a cationic amino acid which is Lysine or Arginine; B and C are hydrophobic amino acids which may be the same or different and are selected from the group consisting of Valine, Leucine, Isoleucine, Tyrosine, Phenylalanine, and Tryptophan; p is an integer with a minimal value of 2; and D is a cationic amino acid or is absent. In one embodiment, A is Lysine, B is Phenylalanine, C is Phenylalanine, D is Lysine, and p is 3. In another embodiment, p is 2-10. In another embodiment, p is 2, 3, 4, 5, 6, 7, 8, 9 or 10.

In other aspects, the cell penetrating molecule Z has the formula: B—X₁—(R—X₂—R)₄, where B is beta-alanine or is absent, X₁ is 6-amino-hexanoic acid or is absent, X₂ is 6-amino-hexanoic acid, and R is arginine or homo-arginine. In some embodiments, R is arginine, selected from the group consisting of L-arginine and D-arginine.

In some aspects, the cell penetrating molecule is a polypeptide having an amino acid sequence selected from the group consisting of SEQ ID NOs: 67-77 (See Table 2).

In some aspects, L is a linker having the formula (Y′)ₙ. In one embodiment, each Y′ is glycine and n is an integer with a minimal value of 2. In some aspects, each Y′ is glycine and n is 2, 3, 4, 5, 6, 7, 8, 9, or 10. In a particular aspect, each Y′ is glycine and n is 2. In another aspect, each Y′ is 8-amino-3,6-dioxaoctnoic acid and n is 1. In another aspect, each Y′ is 5-amino-3-oxapentanoic acid and n is 1. In another aspect, each Y′ is cysteine and n is 1, 2, 3, 4, 5, 6, 7, 8, 9, or 10. In a particular aspect, each Y′ is cysteine and n is 1.

In some embodiments, the PNA-CPP molecule is represented by the formula: N-L-Z, where N comprises a modified backbone. In a particular embodiment, the modified backbone is a PNA backbone.

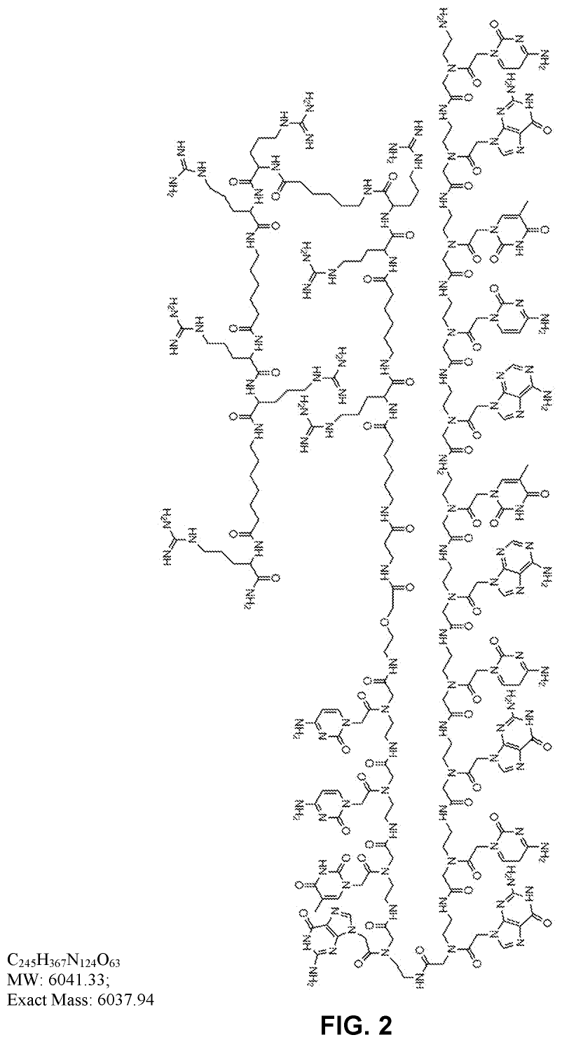

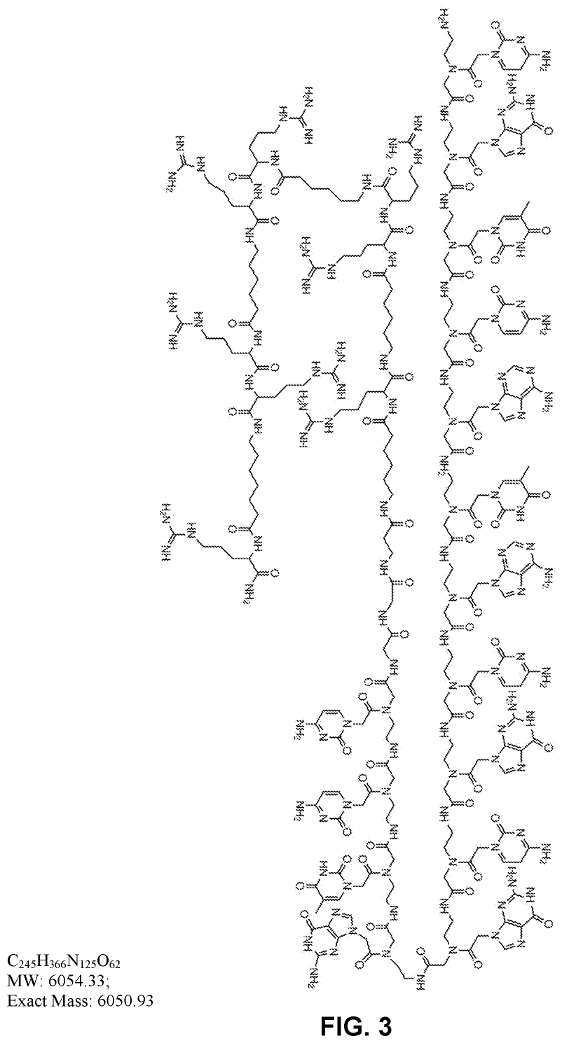

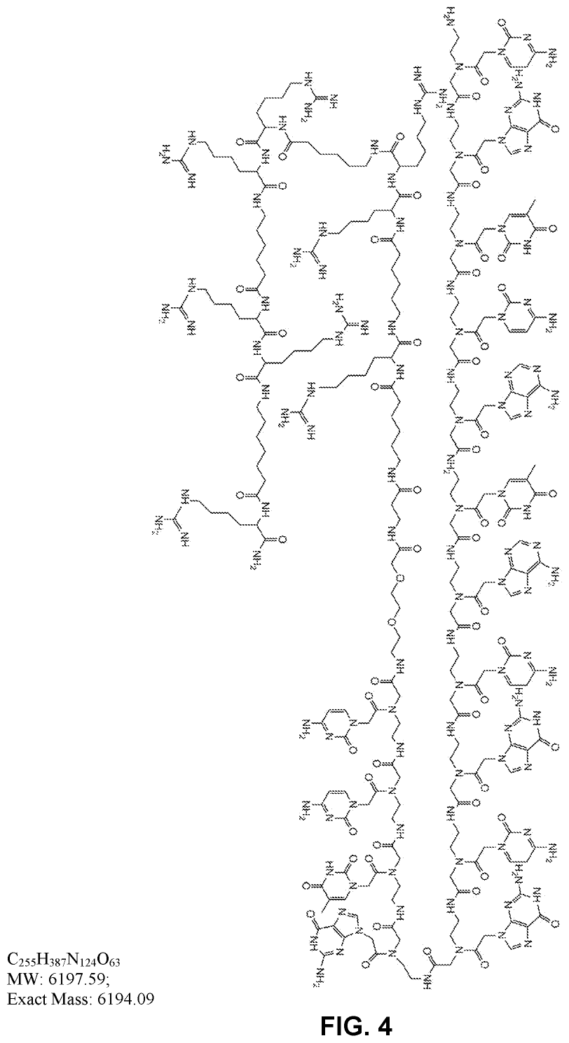

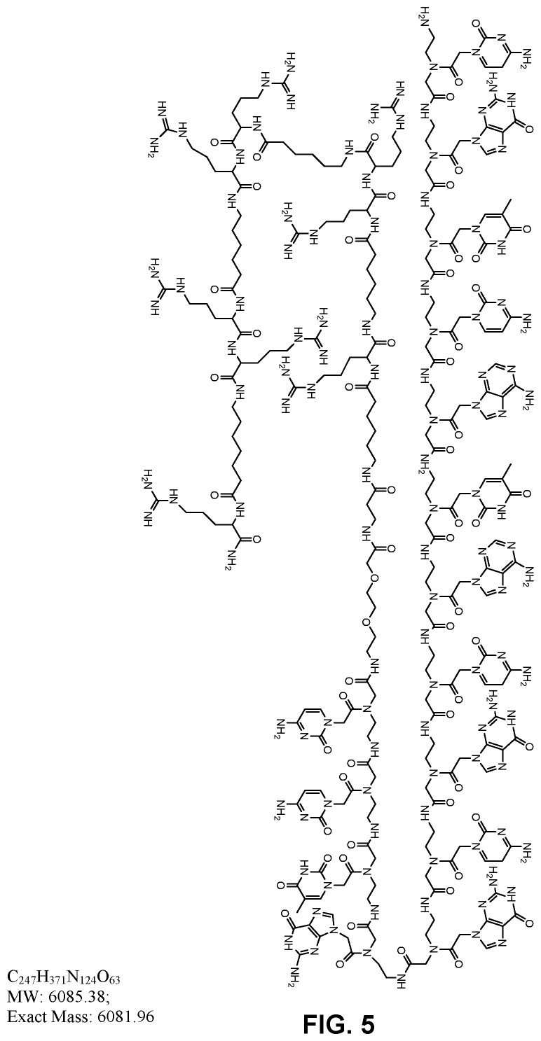

In one embodiment, the PNA-CPP molecule is the compound shown in FIG. 1. In another embodiment, the PNA-CPP molecule is the compound shown in FIG. 2. In another embodiment, the PNA-CPP molecule is the compound shown in FIG. 3. In another embodiment, the PNA-CPP molecule is the compound shown in FIG. 4. In another embodiment, the PNA-CPP molecule is the compound shown in FIG. 5.

### EXAMPLES

### Example 1

### PNA-CPP Derivatives

The PNA-CPP conjugates with linkers shown below in Table 3 were created as described herein:

TABLE 3
Examples of PNA-CPP conjugates

| | | | Cell |
|---|---|---|---|
| Com- | PNA | | penetrating |
| pound | sequence | Linker | molecule |
| I | cgt cat | AEEA | BXRXRRXRRXRRXR |
| | acg cgg | | |
| | tcc | | |
| | (SEQ ID | | |
| | NO: 51) | | |
| II | cgt cat | AEA | BXRXRRXRRXRRXR |
| | acg cgg | | |
| | tcc | | |
| | (SEQ ID | | |
| | NO: 51) | | |
| III | cgt cat | Glycine- | BXRXRRXRRXRRXR |
| | acg cgg | Glycine | |
| | tcc | | |
| | (SEQ ID | | |
| | NO: 51) | | |
| IV | cgt cat | AEEA | BXhRXhRhRXhRhRXhRhRXhR |
| | acg cgg | | |
| | tcc | | |
| | (SEQ ID | | |
| | NO: 51) | | |
| V | cgt cat | AEEA | BXdRXdRdRXdRdRXdRdRXdR |
| | acg cgg | | |
| | tcc | | |
| | (SEQ ID | | |
| | NO: 51) | | |

a-Adenine
g-Guanine
t-Thymine
c-Cytosine
AEEA-8-Amino-3,6-dioxaoctanoic acid
AEA-5-amino-3-oxapentanoic acid
G-Glycine
R-L-Arginine
hR-Homo-Arginine
dR-D-Arginine acid
X-6-amino-hexanoic
B-beta-alanine

The structure of Compounds I-V are shown in FIGS. 1-5, respectively.

### Example 2

### Synthesis of PNA-CPP derivatives

PNA-CPP derivatives of the present disclosure were synthesized by following Merrifield Solid Phase Peptide Synthesis using AAPPTEC automated peptide synthesizers. Each compound was synthesized at a 0.1 micromolar (μM) concentration using Rink-Amide resin in a 50 ml reaction vessel. The Rink-amide resins were deprotected by 20% piperidine in N-Methyl-2-pyrrolidone (NMP). Resins were washed with NMP for 7 times with 2 mins mixing. Three equimolar concentration of Fmoc-amino acids/Fmoc-PNAs were mixed with 2.85 equimolar concentrations of 1-Cyano-2-ethoxy-2-oxoethylidenaminooxy)dimethylamino-morpholino-carbenium hexafluorophosphate in presence of NMP for 1 min and added to the deprotected resins with further addition of 0.3M N,N-Diisopropylethylamine (DIEA) and 0.3M of 2,6 Lutidine for coupling of Fmoc-amino acids/Fmoc-PNAs. Coupling of Fmoc-amino acids/Fmoc-PNAs was performed for 60 mins with continuous shaking and intermittent argon gas bubbling. After coupling, resins were washed four times with NMP with 2 mins mixing. The growing amino acids/PNAs on resins were capped with 1.5 M Acetic anhydride for 30 mins followed by 5 times washing of resins with NMP with 2 mins of shaking. The process of deprotection, coupling, and capping steps repeated till the end of synthesis of compound. After final capping of amino acid/PNA onto growing resins, the final product was deprotected from resins. The crude product was cleaved from resin by 95% trifluoroacetic acid, 2.5% TIS and 2.5 water for 4 hrs at 37° C. The cleavage product was precipitated in 5 volumes of cold ether and the precipitated compound was collected by centrifugation. The ether precipitated compound was air dried for purification.

### Example 3

### Purification of the PNA-CPP Derivatives

The air dried crude compounds were solubilized in 0.1% TFA in HPLC water. The compounds were purified in Waters Prep-150 system. Thirty milligrams of compound was loaded in X-Bridge C18 columns (10 mm×250 mm) with a flow rate of mobile phase 5.5 ml/min. Mobile phase conditions are as follows in Table 4:

TABLE 4
Mobile phase conditions for purification of PNA-CPP conjugates

| Start Time | Solvent A % | Solvent B % |
|---|---|---|
| (min.) | (0.1% TFA in Water) | (0.1% TFA in Acetonitrile) |
| Initial | 100 | 0 |
| 3 | 70 | 30 |
| 15 | 0 | 100 |
| 18 | 0 | 100 |
| 21 | 100 | 0 |
| 24 | 100 | 0 |

The purified fractions were lyophilized and converted to HCl salt or acetate salts. The HCl salts of the compounds were prepared by addition of 10 mM HCl solution into lyophilized compound. The solution was flash freeze and further lyophilized to collect the final compound. Acetate salt of the compounds were converted by passing through the HPLC columns in a 1% acetic acid-water and acetonitrile mobile phase. The purified fractions were lyophilized to collect the acetate salts of the compounds. A small fraction of the compound was used to run in an analytical HPLC column to determine the purity of the compound. The purity of all compounds were >95% (Data not shown). The HCl/acetate salt of compounds were screened for the antimicrobial activities against bacterial strains.

### Example 4

### Assays to Determine the Minimal Inhibitory Concentration (MIC) and Minimal Bactericidal Concentration (MBC) PNA-CPP Derivatives Against S. aureus MRSA Strains

Bioscreen-C instrument was used to detect the MIC and MBC of PNA-CPP derivatives against S. aureus MRSA strains. Different concentration of PNA-CPP derivatives were prepared in 10 mM of sodium bicarbonate buffer pH-7.4 and added to ˜5.0 Log 10 CFUs in a Honey comb Bioscreen-C plate. The plates were incubated in Bioscreen C instrument and growth of bacteria was observed in every 5 mins by measuring the optical density at 420-580 nm with intermittent shaking.

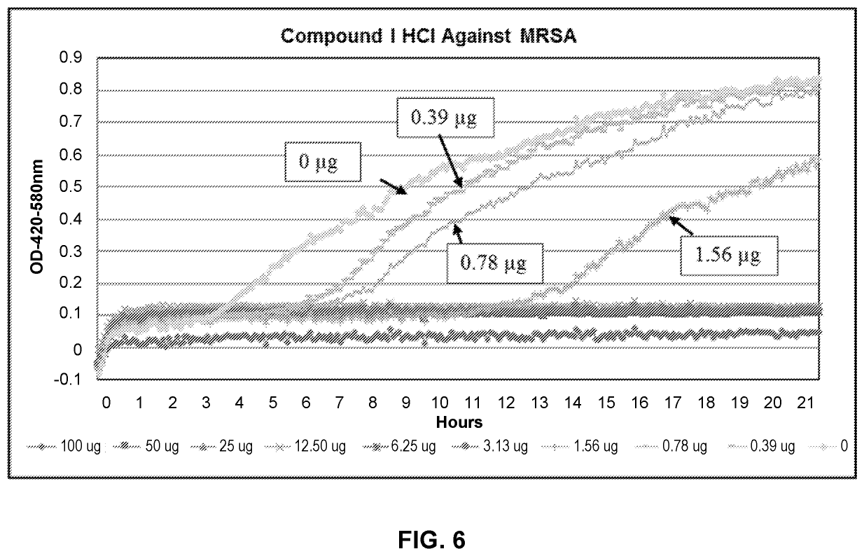

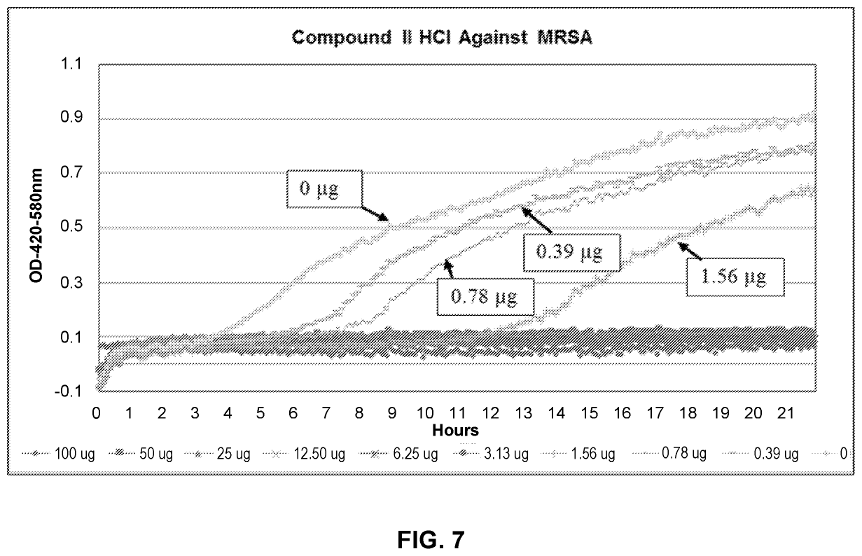

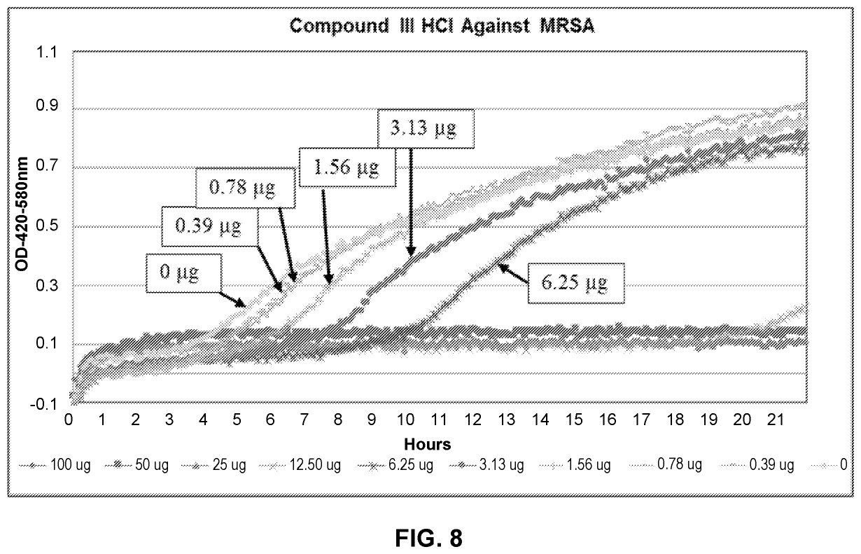

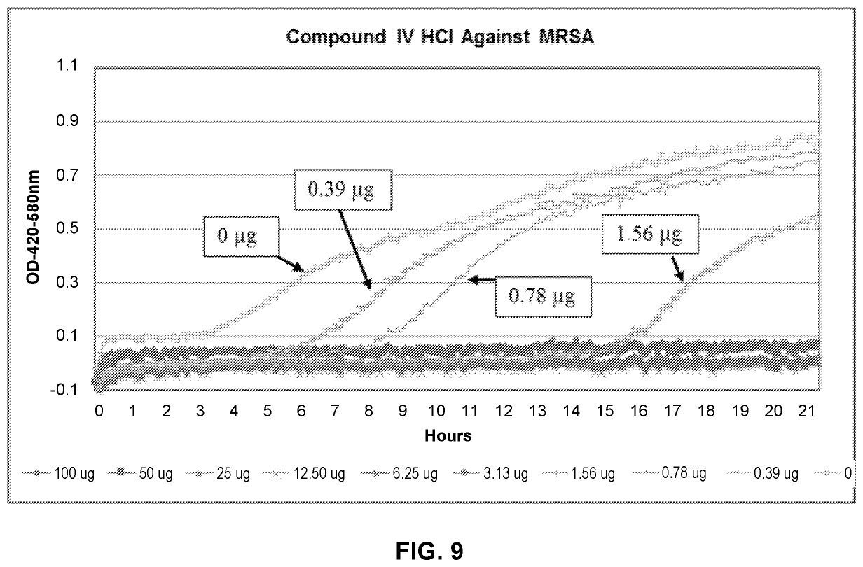

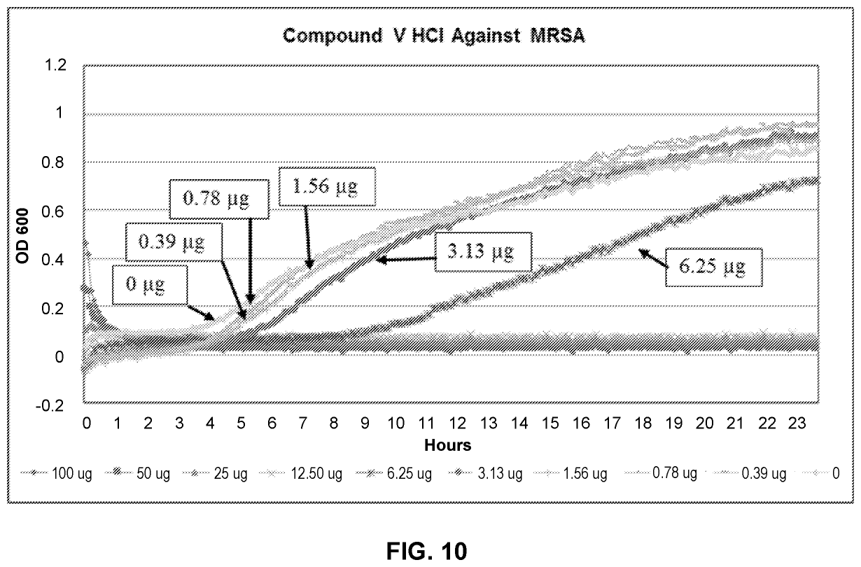

Compound I-V were assayed against MRSA. FIG. 6-10 show a dose response growth curve of MRSA in presence of increasing concentrations of Compound I-HCl (FIG. 6), Compound II-HCl (FIG. 7), Compound III-HCl (FIG. 8), Compound IV-HCl (FIG. 9), and Compound V-HCl (FIG. 10), determined in a Bioscreen-C spectrophotometer. FIG. 6 shows that no MRSA growth was observed in the presence of Compound I at doses from 100 μg to as low as 3.13 μg. Similar results are shown for Compound II in FIG. 7 and Compound IV in FIG. 9. FIG. 8 shows that no MRSA growth was observed in the presence of Compound III at doses from 100 μg to as low as 12.5 μg. Similar results are shown for Compound V in FIG. 10.

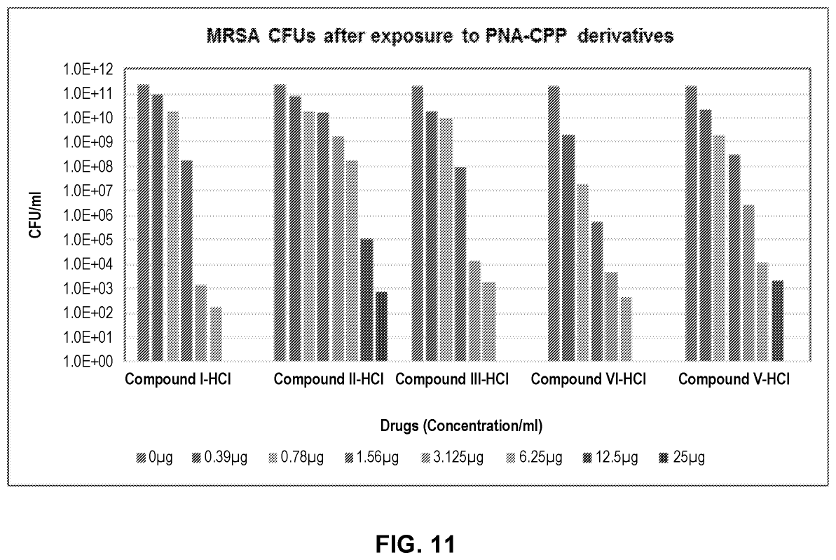

After the growth curve assays, the number of colony forming units (CFU) were counted to determine the MIC and MBC of each PNA-CPP derivative against MRSA. The number of CFUs enumerated are shown in FIG. 11.

Minimum inhibitory concentration (MIC) analyses were performed as described in Clinical and Laboratory Standards Institute, Methods for Dilution Antimicrobial Susceptibility Tests for Bacteria that Grow Aerobically, 7th ed.; Approved Standard M7-A7; CLSI: Wayne, Pa., USA, 2006; volume 26, No. 2. Vancomycin and Oxacillin were used as controls. MIC was determined as the lowest concentration of agent that inhibits bacterial growth detected at A420-580 nm. The results are shown in Table 5.

TABLE 5
MIC and MBC of drugs against the
MRSA clinical isolates and ATCC strains.

| | Compounds MIC | | | |
|---|---|---|---|---|
| | Compound I | Compound II | Vancomycin | Oxacillin |
| Clinical | (∞g) | (∞g) | (∞g) | (∞g) |
| Isolates | MIC/MBC | MIC/MBC | MIC/MBC | MIC/MBC |
| MRSA#1 | 0.78/1.56 | 1.56/3.25 | 5 | 64 |
| MRSA#3 | 1.56/3.25 | 1.56/3.25 | 10 | 64 |
| MRSA#6 | 1.56/1.56 | 1.56/3.25 | 10 | 64 |
| MRSA#15 | 3.25/3.25 | 1.56/3.25 | 5 | 64 |
| MRSA#28 | 3.25/3.25 | 1.56/3.25 | 10 | 128 |
| MRSA#31 | 3.25/3.25 | 1.56/3.25 | 10 | 128 |
| MRSA#37 | 1.56/3.25 | 0.78/1.56 | 10 | 64 |
| MRSA#39 | 3.25/3.25 | 1.56/3.25 | 10 | 64 |
| MRSA#42 | 1.56/1.56 | 0.78/1.56 | 10 | 64 |
| MRSA#43 | 1.56/3.25 | 0.78/1.56 | 10 | 256 |
| USA 300 | 1.56/3.25 | 0.78/1.56 | 10 | 64 |

### Example 5

### Assays to Determine the Minimal Inhibitory Concentration (MIC) and Minimal Bactericidal Concentration (MBC) PNA-CPP Derivatives Against S. aureus MSSA Strains

Bioscreen-C instrument was used to detect the MIC and MBC of PNA-CPP derivatives against S. aureus MSSA strains. Different concentrations of PNA-CPP derivatives were prepared in 10 mM of sodium bicarbonate buffer pH-7.4 and added to ˜5.0 Log 10 CFUs in a Honey comb Bioscreen-C plate. The plates were incubated in Bioscreen C instrument and growth of bacteria was observed in every 5 mins by measuring the optical density at 420-580 nm with intermittent shaking.

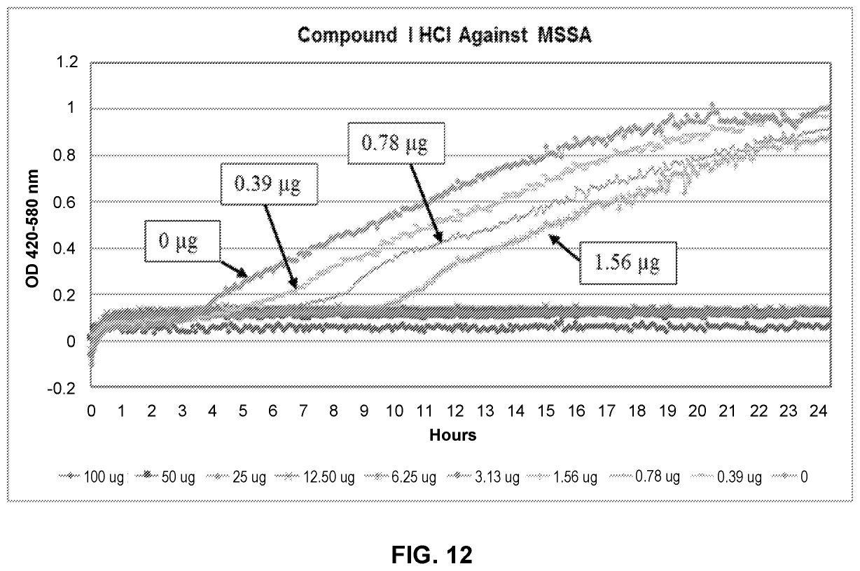

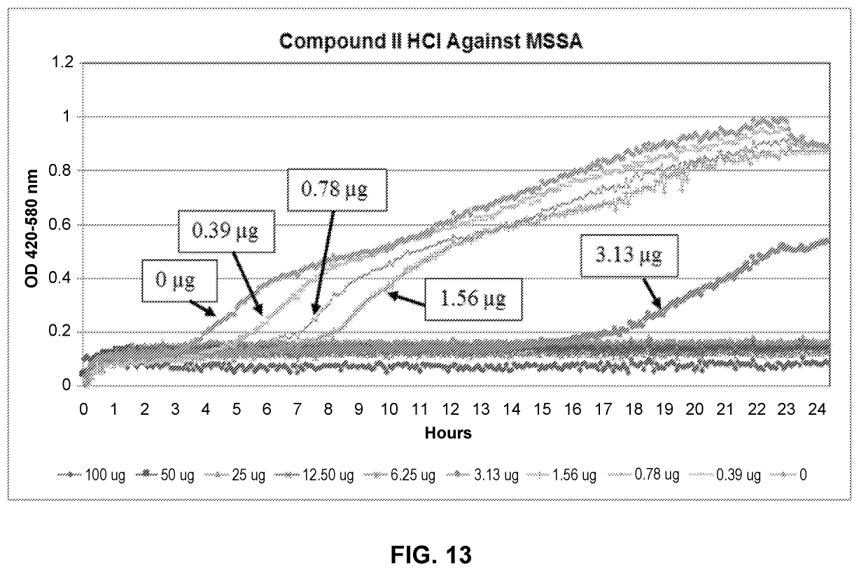

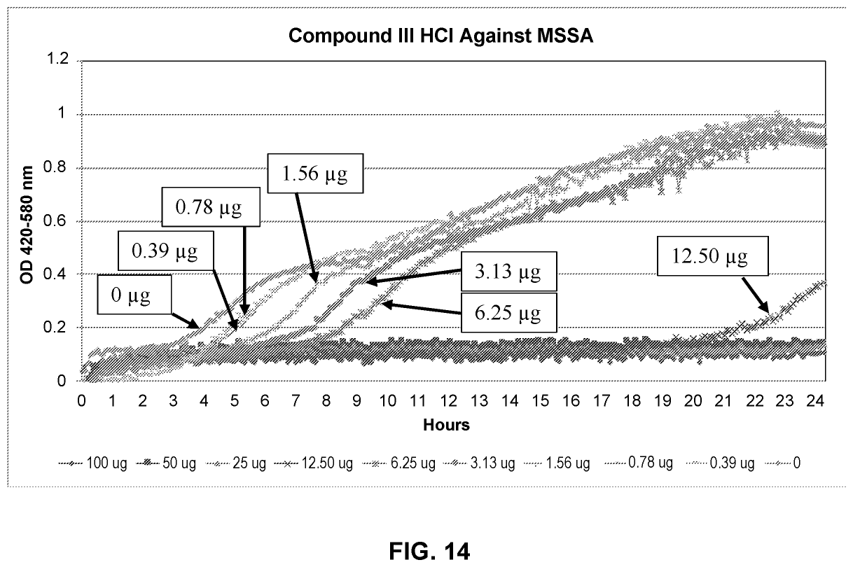

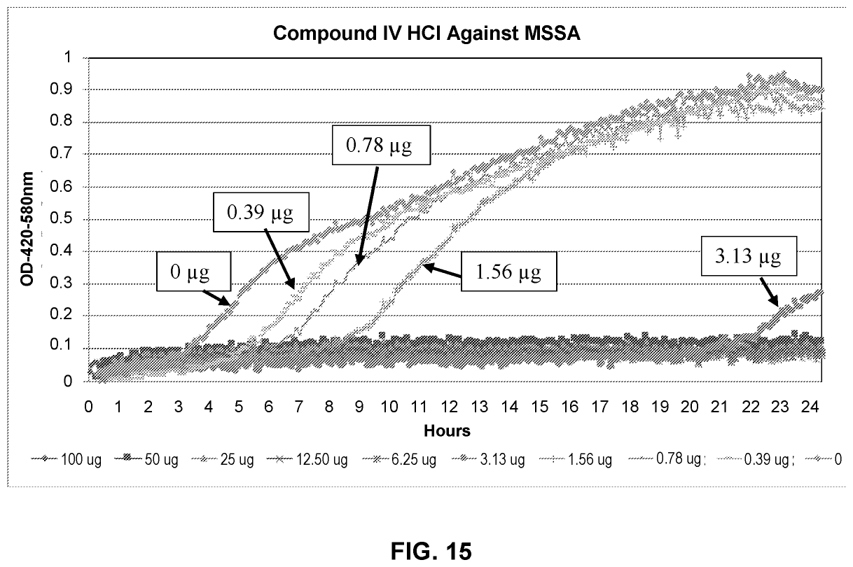

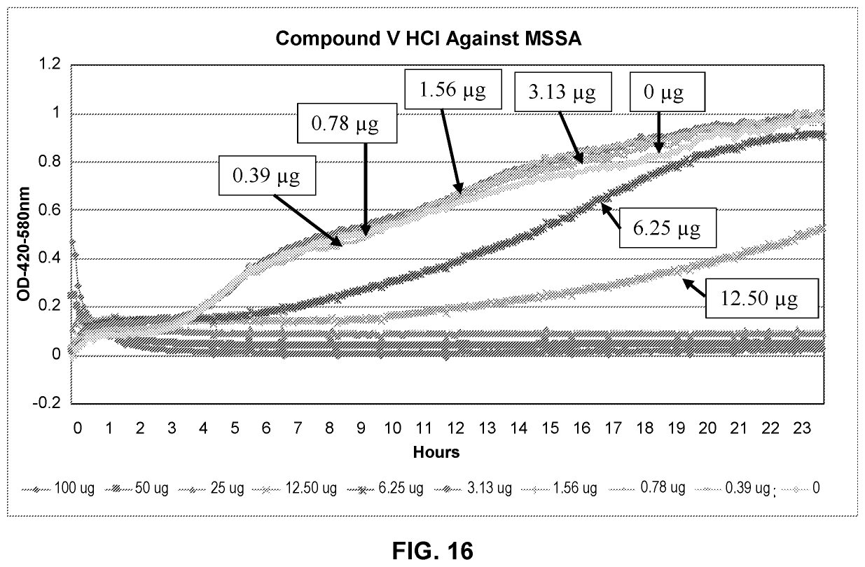

Compound I-V were assayed against MSSA. FIG. 12-16 show a dose response growth curve of MSSA in presence of increasing concentrations of Compound I-HCl (FIG. 12), Compound II-HCl (FIG. 13), Compound III-HCl (FIG. 14), Compound IV-HCl (FIG. 15), and Compound V-HCl (FIG. 16), determined in a Bioscreen-C spectrophotometer. FIG. 12 shows that no MSSA growth was observed in the presence of Compound I at doses from 100 μg down to 3.13 μg. FIG. 13 shows that no MSSA growth was observed in the presence of Compound II at doses from 100 μg down to 6.25 μg. FIG. 14 shows that no MSSA growth was observed in the presence of Compound III at doses from 100 μg down to 25 μg. FIG. 15 shows that no MSSA growth was observed in the presence of Compound IV at doses from 100 μg down to 6.25 μg. FIG. 16 shows that no MSSA growth was observed in the presence of Compound Vat doses from 100 down to 25 μg.

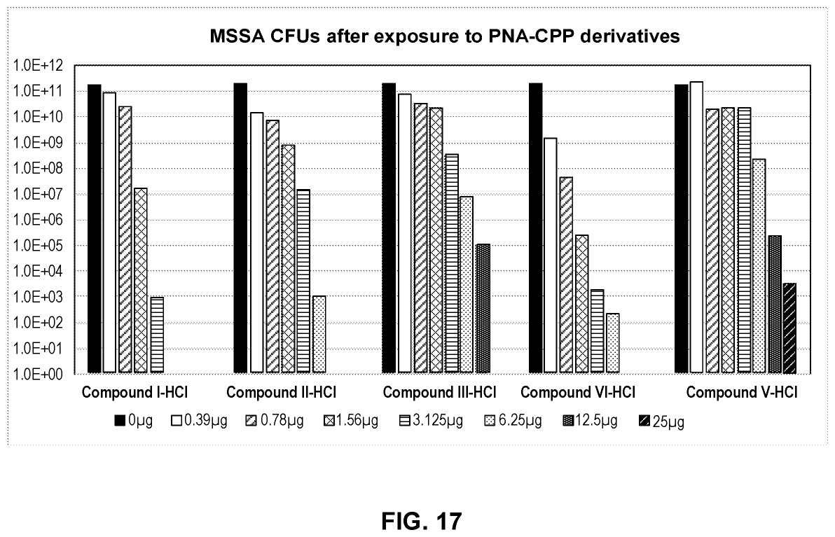

After the growth curve assays, the number of colony forming units (CFU) were counted to determine the MIC and MBC of each PNA-CPP derivative against MSSA. The number of CFUs enumerated are shown in FIG. 17.

Minimum inhibitory concentration (MIC) analyses were performed as described in Clinical and Laboratory Standards Institute, Methods for Dilution Antimicrobial Susceptibility Tests for Bacteria that Grow Aerobically, 7th ed.; Approved Standard M7-A7; CLSI: Wayne, Pa., USA, 2006; volume 26, No. 2. Vancomycin and Oxacillin were used as controls. MIC was determined as the lowest concentration of agent that inhibits bacterial growth detected at A420-580 nm. The results are shown in Table 6.

TABLE 6
MIC and MBC of drugs against
MSSA clinical isolates and ATCC strains

| | Compounds MIC | | | |
|---|---|---|---|---|
| | Compound I | Compound II | Vancomycin | Oxacillin |
| Clinical | (∞g) | (∞g) | (∞g) | (∞g) |
| Isolates | MIC/MBC | MIC/MBC | MIC/MBC | MIC/MBC |
| MRSA#16 | 1.56/1.56 | 1.56/3.25 | 5 | 2 |
| MRSA#25 | 1.56/3.25 | 3.25/3.25 | 10 | 2 |
| MRSA#27 | 0.78/1.56 | 0.78/1.56 | 10 | 2 |
| MRSA#34 | 3.25/3.25 | 3.25/3.25 | 5 | 2 |
| MRSA#38 | 1.56/1.56 | 3.25/3.25 | 10 | 2 |
| MRSA#49 | 1.56/3.25 | 3.25/3.25 | 10 | 2 |
| MRSA#55 | 1.56/3.25 | 3.25/3.25 | 10 | 2 |
| MRSA#57 | 1.56/3.25 | 1.56/3.25 | 20 | 4 |
| MRSA#60 | 0.78/1.56 | 0.78/1.56 | 10 | 2 |
| MRSA#61 | 3.25/3.25 | 0.78/1.56 | 5 | 2 |
| BAA 1721 | 1.56/3.25 | 0.78/1.56 | 10 | <2 |

### Example 6

### Assay to Determine the Emergence of Resistance in S. auereus Against PNA-CPP Derivatives

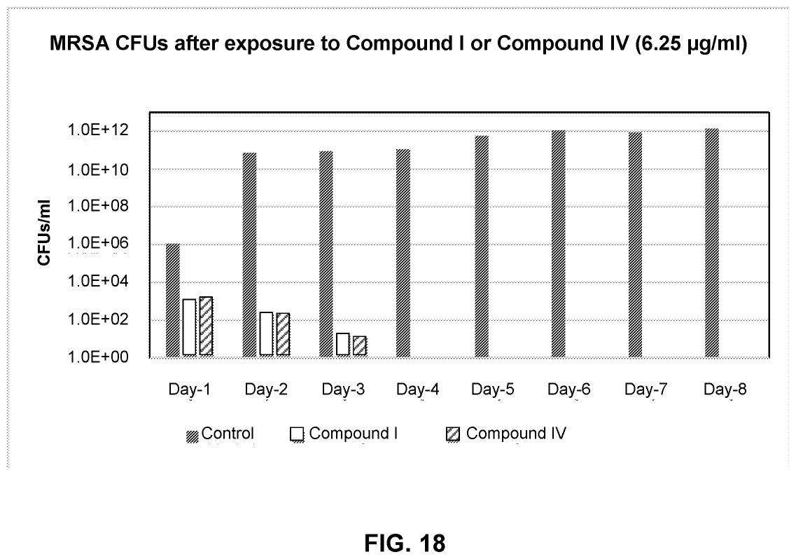

To determine the development of resistance in MRSA and MSSA strains against Compound I-HCl and Compound IV-HCl, an in vitro assay was performed. Twice the amount of MBC of Compound I-HCl (6.25 μg) and Compound IV-HCl (6.25 μg) were mixed with ˜5.0 log 10 CFUs in one ml of Mueller Hinton broth and incubated for 8 days at 37° C. Eight tubes of culture were used for each drug and bacterial strains. At 24 hours of interval, culture tubes were centrifuged and spent media were replaced with fresh media in presence of Compound I-HCl and Compound IV-HCl and further incubated to observe the growth of S. aureus strains. At 24 hours interval, one set of culture tube exposed to drugs were selected and a fraction of the culture used to plate on agar plates to observe the reduction of CFU counts as compared to untreated control. The rest of the culture samples were allowed to grow without any drug for additional 24 hours to observe the growth of S. aureus strains. One set of culture tubes without drug was used as the positive control. The results are shown in FIGS. 18-21.

FIG. 18 shows that each of Compound I and Compound IV reduced MRSA CFUs without emergence of resistant strains through Day 8.

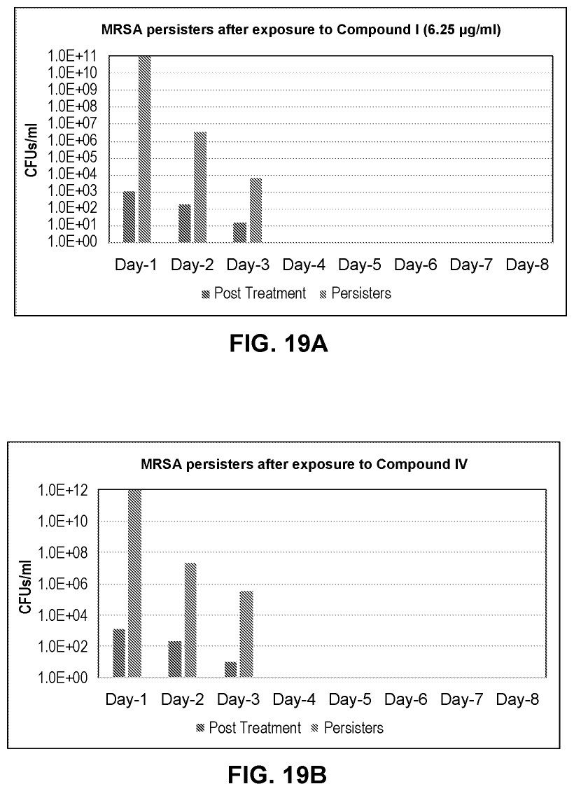

FIG. 19A shows the CFU counts of MRSA strains used to determine the emergence of persister cells after Compound I-HCl treatment. FIG. 19B shows the CFU counts of MRSA strains used to determine the emergence of persister cells after Compound IV-HCl treatment.

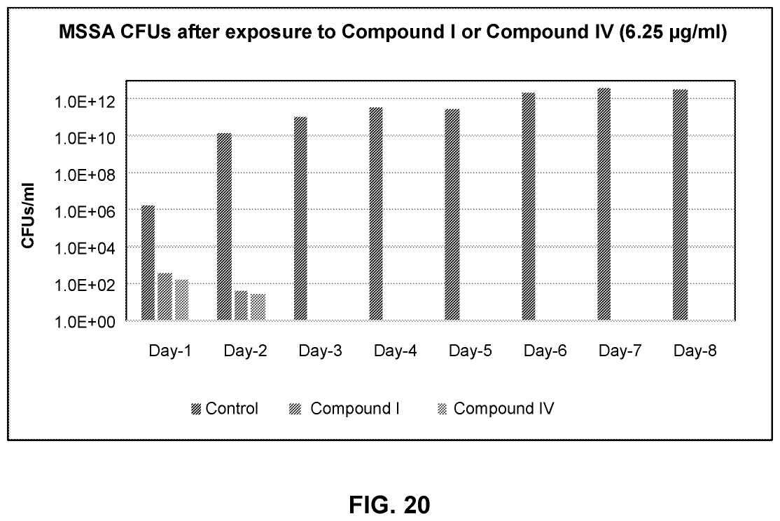

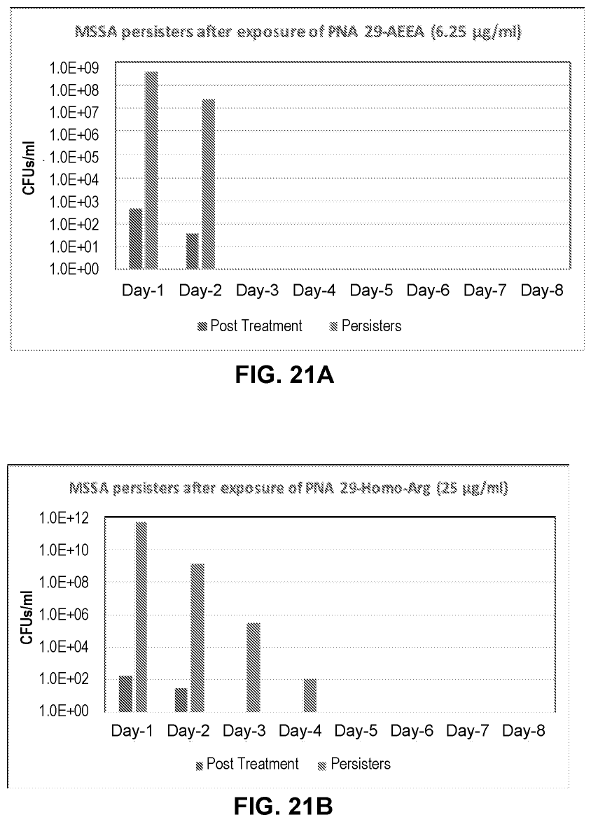

The assays above were also performed using MSSA strains and Compounds I and IV. These results are shown in FIGS. 20-21. FIG. 20 shows that each of Compound I and Compound IV reduced MSSA CFUs without emergence of resistant strains through Day 8.

FIG. 21A shows the CFU counts of MSSA strains used to determine the emergence of persister cells after Compound I-HCl treatment. FIG. 21B shows the CFU counts of MSSA strains used to determine the emergence of persister cells after Compound IV-HCl treatment.

### Example 7

### Single Intravenous Dose Administration to Determine the Maximum Tolerability Dose (MTD) of Drugs in Mice

Single ascending intravenous (IV) dose study was performed to determine tolerability of the drug in mice.

Initially, Compound I-HCl and Compound IV-HCl were administered with a 10 mg/kg intravenous bolus dose using a 10 mL/kg dose volume (0.2 mL) and observed the effects before proceeding to the next higher dose. Both compounds were well tolerated in mice at 10 mg/kg intravenous administration. However, higher concentration (>15 mg/kg) had shown adverse effect and death in mice within 30 mins of administration of the drug.

### Example 8

### Murine Thigh Infection Model to Determine the Antimicrobial Efficacy of Drugs Against S. aureus Infection

Female 5-6 week old CD-1 (18-22 gm) were used in this study. Mice were quarantined for 48 hours before use and housed in groups of 5 with free access to food and water during the study.

The animals were made neutropenic by administration of cyclophosphamide on Days −4 and −1. On Days −4 150 mg/kg of cyclohsphamide administered by intraperitoneal route and 100 mg/kg was administered on Days −1. Days listed are referenced from the date of infection (study day-Day 0).

On Day 0, animals were inoculated intramuscularly (0.1 ml/thigh) with ˜1×105 CFU/mouse of S. aureus (ATCC BAA-1556) into right thigh. One group of mice administered with 10 mg/kg of Compound I at 1 and 13 hrs of post-infection and second group was administered with 10 mg/kg of Compound I at 1, 8, and 17 hrs of post-infection via IV route. Group-3 of mice were administered with vancomycin at 25 mg/kg via subcutaneous route. Group-4 of mice administered with buffer and group-5 used as the inoculation control.

Mice were euthanized by CO₂ inhalation and thighs were removed, and placed in 2 ml of sterile PBS, homogenized, serially diluted and plated to determine the CFU counts. Plates were incubated 18-24 hours and CFUs were counted.

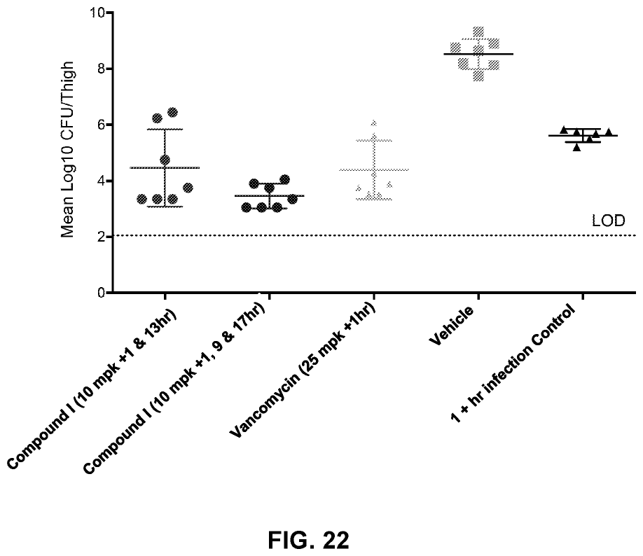

Colony were counted and the number of colonies is converted to CFU/thigh by multiplying the number of colonies by the volume of the thigh homogenate spotted and the dilution at which the colonies were counted (5-50 colonies/spot). All count data were transformed into logo CFU/thigh for calculation of means and standard deviations. Results are shown in FIG. 22 and Table 7.

TABLE 7
CFU counts of MRSA after the S. aureus infection followed by treatment

| | | | | | | | | Mean Log 10 | |
|---|---|---|---|---|---|---|---|---|---|
| | | | | | | | | Change vs. | |
| | Test | Dose | Volume/ | | Time- | Mean Log 10 | Standard | | 24 hr |
| Group | Article | (mg/kg) | Route | Regimen | points | CFU/Thigh | Deviation | 1 hr | (vehicle) |
| 1 | Compound I | 10 | 0.2 ml IV | +1 & 13 hrs | 24 hrs | 4.46 | 1.38 | −1.16 | −4.07 |
| 2 | | 10 | 0.2 ml IV | +1, +9, | | 3.46 | 0.44 | −2.16 | −5.07 |
| | | | | & +17 hrs | | | | | |
| 3 | Vancomycin | 25 | 0.2 ml SC | +1 hr | | 4.39 | 1.05 | −1.23 | −4.14 |
| 4 | Vehicle | 25 | 0.2 ml IV | +1, +9, | | 8.53 | 0.53 | | |
| | | | | & +17 hrs | | | | | |
| 5 | Infection | na | na | na | 1 hr | 5.62 | 0.23 | | |
| | Controls | | | | | | | | |

As shown in Table 7 and FIG. 22, administering Compound I at 1 and 13 hours or at 1, 9, and 17 hours provided comparable or improved reduction in CFU counts compared to Vancomycin positive control treatment.

These results show that the PNA-CPP derivatives of the present disclosure exhibit potent antimicrobial effects against S. aureus infections in vivo.

All patents, patent applications and publications cited herein are fully incorporated by reference.

1. A compound having the formula:
N-L-Z,
or pharmaceutically acceptable salt thereof, wherein
N is an antisense molecule that inhibits the growth of a bacterium comprising a polynucleotide sequence that is antisense to the coding region of a bacterial protein and hybridizes to the coding region under physiological conditions;
L is a linker having the formula (Y′)ₙ, where each Y′ is independently glycine, 8-amino-3,6-dioxaoctanoic acid, or 5-amino-3-oxapentanoic acid, and n is 1 to 6; and
Z is a cell penetrating molecule.

2. The compound of claim 1, wherein N is an antisense molecule that inhibits the growth of Staphylococcus aureus comprising a polynucleotide sequence that is antisense to the coding region of a Staphylococcus aureus ribosomal protein or membrane stability protein.

3. The compound of claim 1, wherein N has a sequence selected from the group consisting of SEQ ID NOs: 1-66.

4. The compound of claim 1, wherein Z has the formula: (ABC)ₚ-D, wherein
A is a cationic amino acid which is Lysine or Arginine;
B and C are hydrophobic amino acids which may be the same or different and are selected from the group consisting of Valine, Leucine, Isoleucine, Tyrosine, Phenylalanine, and Tryptophan;
p is an integer with a minimal value of 2;
and D is a cationic amino acid or is absent.

5. The compound of claim 4, wherein A is Lysine, B is Phenylalanine, C is Phenylalanine, D is Lysine, and p is 3.

6. The compound of claim 1, wherein Z has the formula:
B—X₁—(R—X₂—R)₄,
where B is beta-alanine or is absent, X₁ is 6-amino-hexanoic acid or is absent, X₂ is 6-amino-hexanoic acid, and R is arginine or homo-arginine.

7. The compound of claim 6, wherein the arginine is selected from the group consisting of L-arginine and D-arginine.

8. The compound of claim 1, wherein Y′ is glycine and n is 2.

9. The compound of claim 1, wherein Y′ is 8-amino-3,6-dioxaoctnoic acid and n is 1.

10. The compound of claim 1, wherein Y′ is 5-amino-3-oxapentanoic acid and n is 1.

11. The compound of claim 1, wherein N has the sequence set forth in SEQ ID NO: 51.

12. The compound of claim 1, wherein N comprises a modified backbone.

13. The compound of claim 12, wherein the modified backbone is a PNA backbone.

14. The compound of claim 1, wherein the compound has the structure set forth in FIG. 1.

15. The compound of claim 1, wherein the compound has the structure set forth in FIG. 2.

16. The compound of claim 1, wherein the compound has the structure set forth in FIG. 3.

17. The compound of claim 1, wherein the compound has the structure set forth in FIG. 4.

18. The compound of claim 1, wherein the compound has the structure set forth in FIG. 5.

19. A pharmaceutical composition comprising the compound of claim 1 and a pharmaceutically acceptable carrier.

20. A method of inhibiting the growth of bacteria, comprising administering the compound of claim 1 or the pharmaceutical composition of claim 19 to a tissue containing said bacteria or suspected of containing said bacteria.

21. A method of treating a bacterial infection, comprising administering to an animal in need thereof an effective amount of the compound of claim 1 or pharmaceutical composition of claim 19.

22. The method of claim 20, wherein the bacteria is a Gram positive bacteria.

23. The method of claim 22, wherein the Gram positive bacteria is methicillin-resistant Staphylococcus aureus (MRSA), methicillin-susceptible Staphylococcus aureus (MSSA), vancomycin-resistant Staphylococcus aureus (“VRSA”), Staphylococcus epidermidis, Bacillus anthracis, Clostridium botulinum, Clostridium dificile, Clostridium perfringens, Clostridium tetani, Corynebacterium diptheriae, vancomycin-resistant Enterococcus spp. (“VRE”), Enterococcus faecalis, Enterococcus faecium, Lysteria monocytogenes, Micrococcus luteus, Mycobacterium leprae, Mycobacterium tuberculosis, Propionibacterium acnes, Streptococcus pneumoniae, Streptococcus pyogenes, or Streptococcus agalactiae.

24. The method of claim 21, wherein the bacteria is a Gram positive bacteria.

25. The method of claim 24, wherein the Gram positive bacteria is methicillin-resistant Staphylococcus aureus (MRSA), methicillin-susceptible Staphylococcus aureus (MSSA), vancomycin-resistant Staphylococcus aureus (“VRSA”), Staphylococcus epidermidis, Bacillus anthracis, Clostridium botulinum, Clostridium dificile, Clostridium perfringens, Clostridium tetani, Corynebacterium diptheriae, vancomycin-resistant Enterococcus spp. (“VRE”), Enterococcus faecalis, Enterococcus faecium, Lysteria monocytogenes, Micrococcus luteus, Mycobacterium leprae, Mycobacterium tuberculosis, Propionibacterium acnes, Streptococcus pneumoniae, Streptococcus pyogenes, or Streptococcus agalactiae.


SEQUENCE LISTING (131 sequences)

SEQ ID NO: 1 - DNA, 15 bp, Artificial Sequence
  synthetic antisense strand
tttcatttcggcacc

SEQ ID NO: 2 - DNA, 15 bp, Artificial Sequence
  synthetic antisense strand
cgctcacttttgtaa

SEQ ID NO: 3 - DNA, 12 bp, Artificial Sequence
  synthetic antisense strand
acgaggcataat

SEQ ID NO: 4 - DNA, 12 bp, Artificial Sequence
  synthetic antisense strand
tgtttacccata

SEQ ID NO: 5 - DNA, 12 bp, Artificial Sequence
  synthetic antisense strand
aacatcggaatg

SEQ ID NO: 6 - DNA, 12 bp, Artificial Sequence
  synthetic antisense strand
actcgtggcata

SEQ ID NO: 7 - DNA, 12 bp, Artificial Sequence
  synthetic antisense strand
cgagccataata

SEQ ID NO: 8 - DNA, 12 bp, Artificial Sequence
  synthetic antisense strand
acgcataataat

SEQ ID NO: 9 - DNA, 12 bp, Artificial Sequence
  synthetic antisense strand
ttacgtgccatt

SEQ ID NO: 10 - DNA, 12 bp, Artificial Sequence
  synthetic antisense strand
tacgtgccatat

SEQ ID NO: 11 - DNA, 12 bp, Artificial Sequence
  synthetic antisense strand
cgagccatgtat

SEQ ID NO: 12 - DNA, 12 bp, Artificial Sequence
  synthetic antisense strand
tcatgttatggc

SEQ ID NO: 13 - DNA, 12 bp, Artificial Sequence
  synthetic antisense strand
gttggatcatta

SEQ ID NO: 14 - DNA, 12 bp, Artificial Sequence
  synthetic antisense strand
tctttcgctcac

SEQ ID NO: 15 - DNA, 12 bp, Artificial Sequence
  synthetic antisense strand
tacgagccattt

SEQ ID NO: 16 - DNA, 12 bp, Artificial Sequence
  synthetic antisense strand
tagccattgtcg

SEQ ID NO: 17 - DNA, 12 bp, Artificial Sequence
  synthetic antisense strand
catcgaaagtcc

SEQ ID NO: 18 - DNA, 15 bp, Artificial Sequence
  synthetic antisense strand
gttctcattttatat

SEQ ID NO: 19 - DNA, 15 bp, Artificial Sequence
  synthetic antisense strand
tagccacgatgtgca

SEQ ID NO: 20 - DNA, 15 bp, Artificial Sequence
  synthetic antisense strand
ttagccatttatagt

SEQ ID NO: 21 - DNA, 15 bp, Artificial Sequence
  synthetic antisense strand
agacattcagacacc

SEQ ID NO: 22 - DNA, 15 bp, Artificial Sequence
  synthetic antisense strand
gttggcatgtgatat

SEQ ID NO: 23 - DNA, 15 bp, Artificial Sequence
  synthetic antisense strand
tttacgaggcataat

SEQ ID NO: 24 - DNA, 15 bp, Artificial Sequence
  synthetic antisense strand
tactgccatgatata

SEQ ID NO: 25 - DNA, 15 bp, Artificial Sequence
  synthetic antisense strand
tgatttgtcattata

SEQ ID NO: 26 - DNA, 15 bp, Artificial Sequence
  synthetic antisense strand
tatactcattttggg

SEQ ID NO: 27 - DNA, 15 bp, Artificial Sequence
  synthetic antisense strand
aaattgccataatca

SEQ ID NO: 28 - DNA, 15 bp, Artificial Sequence
  synthetic antisense strand
tttagacatctgtat

SEQ ID NO: 29 - DNA, 15 bp, Artificial Sequence
  synthetic antisense strand
taacatcggaatgca

SEQ ID NO: 30 - DNA, 15 bp, Artificial Sequence
  synthetic antisense strand
cagtaatcataataa

SEQ ID NO: 31 - DNA, 15 bp, Artificial Sequence
  synthetic antisense strand
agcaaacatactttg

SEQ ID NO: 32 - DNA, 15 bp, Artificial Sequence
  synthetic antisense strand
gagccataataagac

SEQ ID NO: 33 - DNA, 15 bp, Artificial Sequence
  synthetic antisense strand
ttgacgcataataat

SEQ ID NO: 34 - DNA, 15 bp, Artificial Sequence
  synthetic antisense strand
tttacgtgccattta

SEQ ID NO: 35 - DNA, 15 bp, Artificial Sequence
  synthetic antisense strand
tacgtgccatattaa

SEQ ID NO: 36 - DNA, 15 bp, Artificial Sequence
  synthetic antisense strand
tttagccataactag

SEQ ID NO: 37 - DNA, 15 bp, Artificial Sequence
  synthetic antisense strand
cgagccatgtatttg

SEQ ID NO: 38 - DNA, 15 bp, Artificial Sequence
  synthetic antisense strand
gatcatttcaatact

SEQ ID NO: 39 - DNA, 15 bp, Artificial Sequence
  synthetic antisense strand
actcatgttatggca

SEQ ID NO: 40 - DNA, 15 bp, Artificial Sequence
  synthetic antisense strand
tttagccacttaatt

SEQ ID NO: 41 - DNA, 15 bp, Artificial Sequence
  synthetic antisense strand
cggttcaaagtggga

SEQ ID NO: 42 - DNA, 15 bp, Artificial Sequence
  synthetic antisense strand
tggatcattagttaa

SEQ ID NO: 43 - DNA, 15 bp, Artificial Sequence
  synthetic antisense strand
ggtagtaacattatt

SEQ ID NO: 44 - DNA, 15 bp, Artificial Sequence
  synthetic antisense strand
ttgacccacagtatt

SEQ ID NO: 45 - DNA, 15 bp, Artificial Sequence
  synthetic antisense strand
ttccattaggatgtc

SEQ ID NO: 46 - DNA, 15 bp, Artificial Sequence
  synthetic antisense strand
gagccatttgggcgc

SEQ ID NO: 47 - DNA, 15 bp, Artificial Sequence
  synthetic antisense strand
agccattgtcgctta

SEQ ID NO: 48 - DNA, 15 bp, Artificial Sequence
  synthetic antisense strand
ttccattatccgagc

SEQ ID NO: 49 - DNA, 15 bp, Artificial Sequence
  synthetic antisense strand
ggtcatcgaaagtcc

SEQ ID NO: 50 - DNA, 15 bp, Artificial Sequence
  synthetic antisense strand
gttttaccatgcaaa

SEQ ID NO: 51 - DNA, 15 bp, Artificial Sequence
  synthetic antisense strand
cgtcatacgcggtcc

SEQ ID NO: 52 - DNA, 15 bp, Artificial Sequence
  synthetic antisense strand
atccatcgtaaatcc

SEQ ID NO: 53 - DNA, 12 bp, Artificial Sequence
  synthetic antisense strand
cattactacgca

SEQ ID NO: 54 - DNA, 12 bp, Artificial Sequence
  synthetic antisense strand
tttcgtcattaa

SEQ ID NO: 55 - DNA, 12 bp, Artificial Sequence
  synthetic antisense strand
tcatacgcggtc

SEQ ID NO: 56 - DNA, 12 bp, Artificial Sequence
  synthetic antisense strand
ccgacatattac

SEQ ID NO: 57 - DNA, 12 bp, Artificial Sequence
  synthetic antisense strand
catcgtaaatcc

SEQ ID NO: 58 - DNA, 15 bp, Artificial Sequence
  synthetic antisense strand
tgcatccaaactgaa

SEQ ID NO: 59 - DNA, 15 bp, Artificial Sequence
  synthetic antisense strand
attcatattcggtca

SEQ ID NO: 60 - DNA, 15 bp, Artificial Sequence
  synthetic antisense strand
acaaaaatcataact

SEQ ID NO: 61 - DNA, 15 bp, Artificial Sequence
  synthetic antisense strand
ttaaacatggtcttt

SEQ ID NO: 62 - DNA, 15 bp, Artificial Sequence
  synthetic antisense strand
tactcattttatcaa

SEQ ID NO: 63 - DNA, 15 bp, Artificial Sequence
  synthetic antisense strand
gattttcgtcattaa

SEQ ID NO: 64 - DNA, 15 bp, Artificial Sequence
  synthetic antisense strand
agtgtgtcattatat

SEQ ID NO: 65 - DNA, 15 bp, Artificial Sequence
  synthetic antisense strand
gtctcatgtgtttcc

SEQ ID NO: 66 - DNA, 15 bp, Artificial Sequence
  synthetic antisense strand
tgtcatttcgttttc

SEQ ID NO: 67 - protein, 10 aa, Artificial Sequence
  peptide
KFFKFFKFFK

SEQ ID NO: 68 - protein, 10 aa, Artificial Sequence
  peptide
RFFRFFRFFR

SEQ ID NO: 69 - protein, 23 aa, Artificial Sequence
  peptide
GIGKWLHSAKKFGKAFVGEIMNS

SEQ ID NO: 70 - protein, 21 aa, Artificial Sequence
  peptide
AGYLLGKINLKALAALAKKIL

SEQ ID NO: 71 - protein, 6 aa, Artificial Sequence
  peptide
KKLWLW

SEQ ID NO: 72 - protein, 8 aa, Artificial Sequence
  peptide
RRKWLWLW

SEQ ID NO: 73 - protein, 8 aa, Artificial Sequence
  peptide
KQRWLWLW

SEQ ID NO: 74 - protein, 18 aa, Artificial Sequence
  peptide
LLIILRRRIRKQAHAHSK

SEQ ID NO: 75 - protein, 16 aa, Artificial Sequence
  peptide
RQIKIWFQNRRMKWKK

SEQ ID NO: 76 - protein, 12 aa, Artificial Sequence
  peptide
GRKKRRQRRRPQ

SEQ ID NO: 77 - protein, 13 aa, Artificial Sequence
  peptide
ILPWKWPWWPWRR

SEQ ID NO: 78 - DNA, 15 bp, Artificial Sequence
  peptide
ttttccatgatttat

SEQ ID NO: 79 - DNA, 15 bp, Artificial Sequence
  noncoding peptide
aacattttggttttt

SEQ ID NO: 80 - DNA, 12 bp, Artificial Sequence
  peptide
tccatgatttat

SEQ ID NO: 81 - DNA, 501 bp, Staphylococcus aureus
atgtctgctatcattgaagctaaaaaacaactagttgatgaaattgctgaggtactatcaaattcagtttcaacagtaatcgttgactatcgtggattaacagtagctgaagttactgacttacgttcacaattacgtgaagctggtgttgagtataaagtatacaaaaacactatggtacgtcgtgcagctgaaaaagctggtatcgaaggcttagatgaattcttaacaggtcctactgctattgcaacttcaagtgaagatgctgtagctgcagcgaaagtaatttctggatttgctaaagatcatgaagcattagaaattaaatcaggtgttatggaaggcaatgttattacagcagaagaagttaaaactgttggttcattaccttcacacgatggtcttgtatctatgcttttatcagtattacaagctcctgtacgcaacttcgcttatgcggttaaagctattggagaacaaaaagaagaaaacgctgaataa

SEQ ID NO: 82 - DNA, 423 bp, Staphylococcus aureus
gtggctaaaaaagtagataaagttgttaaattacaaattcctgcaggtaaagcgaatccagcaccaccagttggtccagcattaggtcaagcaggtgtgaacatcatgggattctgtaaagagttcaatgcacgtactcaagatcaagcaggtttaattattccggtagaaatcagtgtttatgaagatcgttcatttacatttattacaaaaactccaccggctccagtattacttaaaaaagcagctggtattgaaaaaggttcaggcgaaccaaacaaaactaaagttgctacagtaactaaagatcaagtacgcgaaattgctaacagcaaaatgcaagacttaaacgctgctgacgaagaagcagctatgcgtattatcgaaggtactgcacgtagtatgggtatcgttgtagaataa

SEQ ID NO: 83 - DNA, 438 bp, Staphylococcus aureus
atgcgtcaaacatttatggcaaatgaatcaaacattgagcgcaaatggtatgttatcgatgctgaaggccaaacattaggtcgtttatcatcagaagtagcatctatcttacgcggtaaaaataaagtaacttacacaccacacgttgatactggtgattatgtaatcgttattaatgcatcaaaaatcgaatttactggtaacaaagaaactgacaaagtttactaccgtcactcaaatcatccaggtggtatcaaatcaatcactgctggtgaattaagaagaactaacccagaacgtttaattgaaaactcaattaaaggtatgttaccaagcactcgtttaggcgaaaaacaaggtaaaaaattatttgtatatggtggcgctgaacatccacacgctgcacaacaaccagaaaactacgaattacgtggttaa

SEQ ID NO: 84 - DNA, 369 bp, Staphylococcus aureus
atgatccaacaagaaacacgcttgaaagtagcagacaactctggtgctcgtgaagttcttacaatcaaagtattaggtggatctggtcgtaaaacagcaaacatcggcgatgttatcgtatgtactgttaaaaatgcaacaccaggtggcgttgttaaaaaaggtgacgttgtcaaagctgtaatcgtacgtactaagtcaggtgttcgtcgtaatgacggttcatacatcaaatttgatgaaaatgcatgtgttatcatccgtgatgacaaaggcccacgtggtactcgtatcttcggacctgttgctcgtgaattacgtgaaggtaacttcatgaaaatcgtatcattagcaccagaagtactttaa

SEQ ID NO: 85 - DNA, 441 bp, Staphylococcus aureus
atgaaattacatgagttaaaaccggcagaaggttcacgtaaagaacgcaatcgtgttggacgtggtgttgcgacaggtaatggtaaaacaagtggtcgcggacacaaaggtcaaaaagctcgttcaggcggtggtgtaagaccaggatttgaaggtggtcaattaccattattccgtcgtttaccaaaacgtggttttactaacataaatcgtaaagaatatgctattgttaacttagaccaacttaataaatttgaagatggtactgaagtaactccagctttattagtagaatctggtgttgttaagaatgaaaaatctggtatcaaaatactaggtaatggttcacttgataagaaattgacagtgaaagctcataaattctcagcttcagcagcagaagctattgatgctaaaggtggagcacacgaggtgatctaa

SEQ ID NO: 86 - DNA, 434 bp, Staphylococcus aureus
atgttactaccaaaacgtgtaaaatatcgtcgtcaacatcgtcctaaaacaactggtcgttctaaaggcggtaactacgtaacatttggtgagtttggtttacaagctacaacaacgtcttggatcacatctcgtcaaatcgaatctgctcgtatagcaatgacacgttacatgaaacgtggcgggaaagtttggattaaaatcttcccacatacaccatatactaaaaaacctttagaagtacgtatgggtgctggtaaaggtgcggttgaaggctggatcgcagttgttaaaccaggtagaattttattcgaagttgctggcgtttctgaagagttgcgcgtgaagcactacgtttagcaagtcacaaacttccagtaaaaactaagtttgtaaaacgtgaggaattgggtggtgaaacaaatgaaagctaa

SEQ ID NO: 87 - DNA, 360 bp, Staphylococcus aureus
atgatcagtaaaattgataaaaataaagtgcgtttaaaaagacatgctcgtgttcgtactaacttatcaggtacagctgaaaagccacgtttaaacgtatatcgttcaaacaagcatatctacgctcaaattattgatgataataaaggcgtaacattagctcaagcttcttcaaaagacagcgacattgctactacagcaactaaagttgaattagcaactaaagtcggtgaagcaattgctaaaaaagctgctgacaaaggcattaaagaaatcgtatttgaccgtggaggatatttatatcacggacgtgttaaagcattagctgaagcagcaagagaaagcggattagaattttaa

SEQ ID NO: 88 - DNA, 351 bp, Staphylococcus aureus
atgacaaatcacaaattaatcgaagcagtaactaaatcacaattgcgtacagacttaccaagtttccgtcctggtgatactttacgtgtacacgtacgtatcattgagggtactcgtgagcgtatccaagtattcgaaggcattgtaattaaacgtcgtggcggtggcgtttctgaaacgtttacagttcgtaaaatttcatcaggtgttggcgtggaacgtacattcccattacacacaccaaaaattgaaaaaatcgaagttaaacgtcgtggtaaagtacgtcgtgctaaattatattacttacgtagtttacgtggtaaagctgctagaatccaagaaattcgttaa

SEQ ID NO: 89 - DNA, 693 bp, Staphylococcus aureus
atggctaaaaaaggtaaaaagtatcaagaagcagctagtaaagttgaccgtactcagcactacagtgttgaagaagcaattaaattagctaaagaaacaagcattgctaactttgacgcttctgttgaagttgcattccgtttaggaattgatacacgtaaaaatgaccaacaaatccgtggtgcagttgtattaccaaacggaactggtaaatcacaaagtgtattagtattcgctaaaggtgacaaaattgctgaagctgaagcagcaggtactgactatgtaggtgaagcagaatacgttcaaaaaatccaacaaggttggttcgacttcgatgtagtagttgctacaccagacatgatgggtgaagttggtaaattaggtcgtgtattaggaccaaaaggtttaatgccaaaccctaaaactggaactgtaacaatggatgttaaaaaagctgttgaagaaatcaaagctggtaaagtagaatatcgtgctgaaaaagctggtatcgtacatgcatcaattggtaaagtttcatttactgatgaacaattaattgaaaacttcaatactttacaagatgtattagctaaagctaaaccatcatctgctaaaggtacatacttcaaatctgttgctgtaactacaacaatgggtcctggagttaaaattgatactgcaagtttcaaataa

SEQ ID NO: 90 - DNA, 357 bp, Staphylococcus aureus
atgccacgagttaaaggtggaacagtaacaagagcgcgtcgtaaaaaaacgattaaattagctaaaggttacttcggttcaaaacatacattatacaaagtagctaagcaacaagtaatgaaatcaggtcaatatgctttccgtgaccgtcgtcaacgtaaacgtgacttccgtaaattatggattacacgtatcaacgcagcagctcgtcaacatgaaatgagctactcacgtttaatgaacggtttgaaaaaagctggtatcgacattaaccgtaaaatgttatcagaaatcgcaatttctgacgaaaaagcatttgctcaattagtaactaaagctaaagatgctttaaaataa

SEQ ID NO: 91 - DNA, 309 bp, Staphylococcus aureus
atgtttgctattattgaaacaggtggaaaacaaatcaaagtagaagaaggtcaagaaatcttcgttgaaaaattagacgtaaacgaaggagatacttttacatttgataaagtattatttgtaggtggagattcagttaaagttggagcgccaacagttgaaggtgcaacagttactgctactgttaataaacaaggtcgcggtaaaaaaatcactgtattcacatacaaacgtcgtaaaaattcaaaacgtaaaaaaggccatcgtcaaccatacactaaattaacaatcgataaaatcaacgcgtaa

SEQ ID NO: 92 - DNA, 354 bp, Staphylococcus aureus
atggaagcaaaagcggttgctagaacaataagaatcgcacctcgtaaagtaagactagttcttgacttaatcagaggtaaaaatgctgctgaagctattgcaattttaaaattaacaaacaaagcttcatcaccagtaattgaaaaagtattaatgtccgctttagctaatgctgaacataactatgacatgaacacagatgaattagtagttaaagaagcatatgctaacgaaggaccaacattaaaacgtttccgtccacgtgcgcaaggtcgtgcaagtgcgattaacaaacgtacaagccacattacaatcgtcgtaagtgacggtaaagaagaagctaaagaagcttaa

SEQ ID NO: 93 - DNA, 276 bp, Staphylococcus aureus
atggaagcaagagatattcttaagcgccccgtaatcactgagaaatcttctgaagcaatggctgaagacaaatacactttcgacgttgatactcgtgttaacaaaacacaagtaaaaatggcagttgaagaaatcttcaacgtaaaagttgcaagtgttaatatcatgaattacaaacctaagaaaaaacgtatgggccgttaccaaggctatacaaacaaaagaagaaaagcgattgtaactcttaaagaaggatcaatcgacttatttaactaa

SEQ ID NO: 94 - DNA, 285 bp, Staphylococcus aureus
atgttaaaattaaacttacaattcttcgcatctaaaaaaggggtaagttctacaaaaaacggacgtgactctgaatcaaaacgcttaggtgctaaacgtgctgacggtcaattcgtaacaggtggttcaattttatatcgccaacgtggtactaaaatttaccctggtgaaaatgtaggtcgtggtggcgatgatacattattcgctaaaatcgacggcgttgttaaattcgaacgtaaaggtcgcgacaaaaaacaagtttctgtatatgcagtagctgaataa

SEQ ID NO: 95 - DNA, 189 bp, Staphylococcus aureus
atgggtaaacaatgtttcgtaacaggtcgtaaagcttcgactggtaacagacgttcacacgctttaaactctactaaacgtagatggaacgctaaccttcaaaaagttagaatcctagttgacggtaaacctaaaaaagtttgggtttctgcacgtgctttaaaatctggtaaagtaactagagtttaa

SEQ ID NO: 96 - DNA, 834 bp, Staphylococcus aureus
atggctattaaaaagtataagccaataacaaatggtcgtcgtaatatgacttcgttagatttcgcagaaatcacgaaaactacacctgaaaagtcattattaaaaccgctaccgaaaaaagcgggacgtaacaaccaaggtaaattgactgtaagacaccatggtggtggacacaaacgtcaataccgtgttatcgattttaaacgtaacaaagatggtatcaatgcaaaagttgattctattcaatatgatccaaaccgctcagcaaacatcgctttagttgtatatgcagacggtgaaaaacgatatatcattgctcctaaaggattagaagtaggtcaaatcgttgaaagtggtgctgaagctgacatcaaagttggtaacgcattaccattacaaaacattccagttggtacagtagtacacaacatcgagcttaaacctggtaaaggtggacaaatcgctcgttcagctggtgcaagtgctcaagtacttggtaaagaaggtaaatacgtattaatcagattaagatctggtgaagttcgtatgatcttatctacttgccgtgctacaatcggtcaagttggtaacctacaacacgaattagttaacgttggtaaagccggacgttcaagatggaaaggtatccgtccaacagttcgtggttctgtaatgaaccctaacgatcacccacacggtggtggtgaaggtcgtgctcctatcggtagaccatctccaatgtcaccatggggtaaacctacgcttggtaagaaaactcgtcgtggtaaaaaatcatcagacaaacttatcgttcgtggacgtaagaaaaaataa

SEQ ID NO: 97 - DNA, 180 bp, Staphylococcus aureus
atggctaaattacaaattaccctcactcgtagtgttattggtcgtcctgaaacacaacgtaaaactgttgaagctttaggtcttaaaaagactaacagttcagtagttgttgaagataaccctgctattcgtgggcaaatcaacaaagttaagcacttagtaacagtagaagaaaaataa

SEQ ID NO: 98 - DNA, 174 bp, Staphylococcus aureus
atggcagtaccaaaaagaagaacttctaaaactagaaaaaacaaacgtcgtacgcatttcaaaatttcagtaccaggtatgactgaatgcccaaactgtggcgaatacaaattatcacaccgtgtatgtaaaaactgtggttcttacaatggcgaagaagtagcagctaaataa

SEQ ID NO: 99 - DNA, 138 bp, Staphylococcus aureus
atggtaaaacgtacttatcaaccaaataaacgtaaacatagtaaagttcatggtttcagaaaacgcatgagcacaaaaaatggccgtaaagttttagcgcgccgtcgtcgtaaaggccgtaaagttttatctgcataa

SEQ ID NO: 100 - DNA, 663 bp, Staphylococcus aureus
atgaccaaaggaatcttaggaagaaaaattgggatgacacaagtattcggagaaaacggtgaattaatccctgtaacagtagtagaagctaaagaaaatgttgtattacaaaagaaaactgtagaagttgatggatacaacgcaatccaagttggatttgaagacaaaaaagcatacaaaaaagatgcaaaatctaataaatatgctaataaaccagctgaaggtcacgctaaaaaagctgacgcagcacctaagcgcttcattcgtgaattccgcaatgtagacgtggatgcttacgaagtaggtcaagaagtctcagtagatacttttgtagctggcgacgttattgacgtaacaggcgtatcaaaaggtaaaggtttccaaggtgcaattaaacgccacggacaatctcgtggacctatgtcacacggttctcatttccacagagcaccaggttctgtaggtatggcttcagatgcttctagagtatttaaaggccaaaaaatgccaggacgtatgggtggaaacactgtaactgttcaaaacttagaagtagttcaagttgacacagaaaacaaagttatcttagtaaaaggtaacgtacctggacctaaaaaaggtttagtagaaatcagaacttcaattaaaaaaggtaataaataa

SEQ ID NO: 101 - DNA, 540 bp, Staphylococcus aureus
ttgaaccgtttaaaagaaaagtttaacactgaagttactgaaaacttaatgaaaaaattcaattatagttcagtaatggaagtaccaaaaatagataaaatcgttgtgaacatgggtgtaggtgacgcagtacaaaattctaaagtattagacaatgctgttgaagaattagaattgatcactggtcaaaaaccattagtaactaaagctaaaaaatcaatcgcgactttccgtttacgtgaaggtatgccaatcggtgcgaaagtaacacttcgcggtgaaagaatgtatgaattcttagacaaattaatttcagtatcattaccacgtgtacgtgacttccaaggtgtttctaaaaaagcatttgacggacgcggtaactacactttaggtgttaaagaacaattaattttcccagaaatcgactatgataaagtaagtaaagttagaggaatggatattgttatcgtaacgactgctaacactgatgaagaagctcgtgaattgttagctaacttcggtatgccattccgtaaataa

SEQ ID NO: 102 - DNA, 537 bp, Staphylococcus aureus
atgagtcgtgttggtaagaaaattattgacatccctagtgacgtaacagtaacttttgatggaaatcatgtaactgttaaaggtcctaaaggtgaattatcaagaactttaaatgaaagaatgacattcaaacaagaagaaaacacaattgaagttgtaagaccatctgattctaaagaagatagaacaaaccatggtacaactcgtgctttattaaacaatatggtacaaggtgtttctcaaggatacgtaaaagtacttgaacttgttggtgtaggttaccgtgctcaaatgcaaggtaaagacttaatccttaacgttggttattctcacccagtagaaattaaagctgaagaaaacattactttctcagttgagaaaaacacagtcgttaaagttgaaggtatttcaaaagaacaagttggagcattagcatctaacatccgttcagtaagacctccagagccttacaaaggtaaaggtattcgttaccaaggtgaatacgttcgccgtaaagaaggtaaaactggtaaataa

SEQ ID NO: 103 - DNA, 369 bp, Staphylococcus aureus
atggctaatcatgaacaaatcattgaagcgattaaagaaatgtcagtattagaattaaacgacttagtaaaagcaattgaagaagaatttggtgtaactgcagctgctccagtagcagtagcaggtgcagctggtggcgctgacgctgcagcagaaaaaactgaatttgacgttgagttaacttcagctggttcatctaaaatcaaagttgttaaagctgttaaagaagcaactggtttaggattaaaagatgctaaagaattagtagacggagctcctaaagtaatcaaagaagctttacctaaagaagaagctgaaaaacttaaagaacaattagaagaagttggagctactgtagaattaaaataa

SEQ ID NO: 104 - DNA, 390 bp, Staphylococcus aureus
atggcacgtaaacaagtatctcgtaaacgtagagtgaaaaagaatattgaaaatggtgtagcacacatccgttcaacattcaacaacactattgtaactatcactgatgagttcggtaatgctttatcatggtcatcagctggtgcattaggattcaaaggatctaaaaaatcaacaccatttgcagcacaaatggcttctgaaactgcatctaaatcagctatggagcatggtttaaaaacagttgaagtaacagttaaaggacctggtccaggtcgtgaatcagctattcgtgcattacaatctgcaggtttagaagtaactgcgatcagagacgttactccagtacctcataacggttgtcgtccaccaaaacgtcgtcgtgtataa

SEQ ID NO: 105 - DNA, 414 bp, Staphylococcus aureus
atgccaactattaaccaattagtacgtaaaccaagacaaagcaaaatcaaaaaatcagattctccagctttaaataaaggtttcaacagtaaaaagaaaaaatttactgacttaaactcaccacaaaaacgtggtgtatgtactcgtgtaggtacaatgacacctaaaaaacctaactcagcgttacgtaaatatgcacgtgtgcgtttatcaaacaacatcgaaattaacgcatacatccctggtatcggacataacttacaagaacacagtgttgtacttgtacgtggtggacgtgtaaaagacttaccaggtgtgcgttaccatattgtacgtggagcacttgatacttcaggtgttgacggacgtagacaaggtcgttcattatacggaactaagaaacctaaaaactaa

SEQ ID NO: 106 - DNA, 366 bp, Staphylococcus aureus
atggcacgtattgcaggagtagatattccacgtgaaaaacgcgtagttatctcattaacttatatatacggtatcggtacgtcaactgctcaaaaaattcttgaagaagctaacgtatcagctgatactcgtgtgaaagatttaactgatgacgaattaggtcgcatccgtgaagttgtagacggttataaagtcgaaggtgacttacgtcgtgaaactaacttaaatatcaaacgtttaatggaaatttcatcataccgtggtatccgtcaccgtcgtggtttaccagttcgtggtcaaaaaacgaaaaacaacgcgcgtactcgtaaaggaccagttaaaacggtagctaacaagaaaaaataa

SEQ ID NO: 107 - DNA, 186 bp, Staphylococcus aureus
gtggctaaaacttcaatggttgctaagcaacaaaaaaaacaaaaatatgcagttcgtgaatacactcgttgtgaacgttgtggccgtccacattctgtatatcgtaaatttaaattatgccgtatttgtttccgtgaattagcttacaaaggccaaatccctggcgttcgtaaagctagctggtaa

SEQ ID NO: 108 - DNA, 270 bp, Staphylococcus aureus
atggcaatttcacaagaacgtaaaaacgaaatcattaaagaataccgtgtacacgaaactgatactggttcaccagaagtacaaatcgctgtacttactgcagaaatcaacgcagtaaacgaacacttacgtacacacaaaaaagaccaccattcacgtcgtggattattaaaaatggtaggtcgtcgtagacatttattaaactacttacgtagtaaagatattcaacgttaccgtgaattaattaaatcacttggtatccgtcgttaa

SEQ ID NO: 109 - DNA, 264 bp, Staphylococcus aureus
gtgagcgaaagaaacgatcgtaaagtttatgtaggtaaagttgtttcagacaaaatggacaagactattacagtacttgttgaaacttacaaaacacacaaattatacggtaaacgagtaaaatactctaaaaaatacaaaactcatgatgaaaacaattcagctaaattaggagacattgttaaaattcaagaaactcgtcctttatcagcaacaaaacgttttcgtatagtagagattgttgaagagtcagtaattatttaa

SEQ ID NO: 110 - DNA, 279 bp, Staphylococcus aureus
atggctcgtagtattaaaaaaggacctttcgtcgatgagcatttaatgaaaaaagttgaagctcaagaaggaagcgaaaagaaacaagtaatcaaaacatggtcacgtcgttctacaattttccctaatttcatcggacatacttttgcagtatacgacggacgtaaacacgtacctgtatatgtaactgaagatatggtaggtcataaattaggtgagtttgctcctactcgtacattcaaaggacacgttgcagacgacaagaaaacaagaagataa

SEQ ID NO: 111 - DNA, 177 bp, Staphylococcus aureus
atgtctaaaacagtagtacgtaaaaatgaatcacttgaagatgcgttacgtagatttaaacgttcagtttctaaaagtggaacaatccaagaagtacgtaaacgtgaattttacgaaaaaccaagcgtaaaacgtaaaaagaaatcagaagctgcacgtaaacgtaaattcaaataa

SEQ ID NO: 112 - DNA, 654 bp, Staphylococcus aureus
gtgggtcaaaaaattaatccaatcggacttcgtgttggtattatccgtgattgggaagctaaatggtatgctgaaaaagacttcgcttcacttttacacgaagatttaaaaatccgtaaatttattgataatgaattaaaagaagcatcagtttctcacgtagagattgaacgtgctgcaaaccgtatcaacattgcaattcatactggtaaacctggtatggtaattggtaaaggcggttcagaaatcgaaaaattacgcaacaaattaaatgcgttaactgataaaaaagtacacatcaacgtaattgaaatcaaaaaagttgatcttgacgctcgtttagtagctgaaaacatcgcacgtcaattagaaaaccgtgcttcattccgtcgtgtacaaaaacaagcaatcactagagctatgaaacttggtgctaaaggtatcaaaactcaagtatctggtcgtttaggcggagctgacatcgctcgtgctgaacaatattcagaaggaactgttccacttcatacgttacgtgctgacatcgattatgcacacgctgaagctgacactacttacggtaaattaggcgttaaagtatggatctatcgtggagaagttcttcctactaagaacactagtggaggaggaaaataa

SEQ ID NO: 113 - DNA, 603 bp, Staphylococcus aureus
atggctcgattcagaggttcaaactggaaaaaatctcgtcgtttaggtatctctttaagcggtactggtaaagaattagaaaaacgtccttacgcaccaggacaacatggtccaaaccaacgtaaaaaattatcagaatatggtttacaattacgtgaaaaacaaaaattacgttacttatatggaatgactgaaagacaattccgtaacacatttgacatcgctggtaaaaaattcggtgtacacggtgaaaacttcatgattttattagcaagtcgtttagacgctgttgtttattcattaggtttagctcgtactcgtcgtcaagcacgtcaattagttaaccacggtcatatcttagtagatggtaaacgtgttgatattccatcttattctgttaaacctggtcaaacaatttcagttcgtgaaaaatctcaaaaattaaacatcatcgttgaatcagttgaaatcaacaatttcgtacctgagtacttaaactttgatgctgacagcttaactggtactttcgtacgtttaccagaacgtagcgaattacctgctgaaattaacgaacaattaatcgttgagtactactcaagataa

SEQ ID NO: 114 - DNA, 501 bp, Staphylococcus aureus
atggctcgtagagaagaagagacgaaagaatttgaagaacgcgttgttacaatcaaccgtgtagcaaaagttgtaaaaggtggtcgtcgtttccgtttcactgcattagttgtagttggagacaaaaatggtcgtgtaggtttcggtactggtaaagctcaagaggtaccagaagcaatcaaaaaagctgttgaagcagctaaaaaagatttagtagttgttccacgtgttgaaggtacaactccacacacaattactggccgttacggttcaggaagcgtatttatgaaaccggctgcacctggtacaggagttatcgctggtggtcctgttcgtgccgtacttgaattagcaggtatcactgatatcttaagtaaatcattaggatcaaacacaccaatcaacatggttcgtgctacaatcgatggtttacaaaaccttaaaaatgctgaagatgttgcgaaattacgtggcaaaacagtagaagaattatacaattaa

SEQ ID NO: 115 - DNA, 297 bp, Staphylococcus aureus
atgagaacatatgaagttatgtacatcgtacgcccaaacattgaggaagatgctaaaaaagcgttagttgaacgtttcaacggtatcttagctactgaaggtgcagaagttttagaagcaaaagactggggtaaacgtcgcctagcttatgaaatcaatgatttcaaagatggcttctacaacatcgtacgtgttaaatctgataacaacaaagctactgacgaattccaacgtctagctaaaatcagtgacgatatcattcgttacatggttattcgtgaagacgaagacaagtaa

SEQ ID NO: 116 - DNA, 471 bp, Staphylococcus aureus
atgcctcgtaaaggatcagtacctaaaagagacgtattaccagatccaattcataactctaagttagtaactaaattaattaacaaaattatgttagatggtaaacgtggaacagcacaaagaattctttattcagcattcgacctagttgaacaacgcagtggtcgtgatgcattagaagtattcgaagaagcaatcaacaacattatgccagtattagaagttaaagctcgtcgtgtaggtggttctaactatcaagtaccagtagaagttcgtccagagcgtcgtactactttaggtttacgttggttagttaactatgcacgtcttcgtggtgaaaaaacgatggaagatcgtttagctaacgaaattttagatgcagcaaataatacaggtggtgccgttaagaaacgtgaggacactcacaaaatggctgaagcaaacaaagcatttgctcactaccgttggtaa

SEQ ID NO: 117 - DNA, 321 bp, Staphylococcus aureus
atgattactgttgatattacagttaatgatgaaggcaaagtaacagacgttattatggatggccatgctgaccatggtgaatatggtcatgatatcgtttgtgctggagcttcagctgtattgtttggtagtgttaatgcgattataggattgacatctgagagaccagatatcaattatgacgacaatggtggtcattttcatataagaagcgttgatacaaacaacgatgaagcgcaactaattcttcaaacaatgcttgtgtctttacaaactattgaagaagaatataatgagaatattagattaaattataagtga

SEQ ID NO: 118 - DNA, 1149 bp, Staphylococcus aureus
atgtcggataaatattatagatctgcgtatatgaatgtagatttaaacgctgttgcatcaaatttcaaagtattcagtacattgcatccaaataaaacagtgatggctgtcgttaaagccaatgcctatggactaggtagtgttaaagtagcacgtcatttaatggaaaatggcgccacattttttgctgtagcaacgttagatgaagcgatagaacttagaatgcatgggattactgctaaaattttagtcttaggtgtgttaccagctaaagatattgataaagcgatacaacaccgagttgccttaacggttccgtctaaacagtggttgaaagaagcaattaaaaacatttctggtgagcaagagaaaaagttatggttgcacattaaattagatacaggaatgggacgtttaggtattaaagatactaaaacgtatcaagaagtgattgaaatcattcaacaatatgagcaacttgtatttgaaggcgtgtttacacactttgcctgtgctgacgaaccaggagatatgacaactgaacaatatcaacgttttaaagatatggtcaatgaagcaattaaacctgaatatatacattgtcagaactcagcaggctctctattaatggattgccaattctgtaatgcaataagaccaggaatttccctttatggatattatccatcagagtatgtacagcaaaaagttaaagtacaccttaaaccaagtgtgcaattaattgctaatgtagttcaaacaaagacgctacaagcgggtgagtctgtaagttatggtgcaacttatacagctactgacccaactacaatagcattgttacctattggatatgcagatggctatttacgcataatgcaaggtagctttgtaaatgtaaatggtcatcaatgcgaagttattggtcgcgtatgtatggatcagacaattgttaaagtgccagatcaagttaaagctggagattcggtgattttaatagataatcatagagaaagtccacagtcggtagaggtggtagctgaaaagcaacatactattaattatgaagtgctttgtaacttgtcgagacgtttgccgcgaatctatcatgatggtgatcaacgttttgtaacaaatgaattgttaaaataa

SEQ ID NO: 119 - DNA, 2076 bp, Staphylococcus aureus
ttgttaaaaagactaaaagaaaaatcaaatgatgaaatcgttcaaaatacaattaacaagagaattaactttatatttggtgtgattgtatttatttttgcagtactagtactacgtttaggttatttacaaatcgcacaaggctcacattataaacaaattataaaaaatgatgaaaacattacagtaaatgagtctgtgccaagaggtcgtattttagacagaaatgggaaagttttagttgataatgcttctaaaatggctattacatatactaggggtcgaaaaacaacacaatcggaaatgttggatacggctgaaaagttatcaaagctaatcaagatggatactaagaagattacagaacgtgataagaaagatttctggattcagttgcatcctaaaaaagcaaaagcaatgatgacaaaagaacaagctatgttagcagatggaagtattaaacaagatcaatatgataaacaactgttatcgaaaatcggaaaatcacaattagatgaattgtcttctaaagatttacaagttttagccatttttcgagagatgaatgcaggaacagttttagatccacaaatgataaaaaatgaagatgtcagtgaaaaagagtatgcagcagtttctcagcaactttccaaattaccaggtgttaacacgtctatggattgggatagaaaatatccatatggcgacactttaagaggtatatttggagatgtatcgacacctgctgaaggtattccaaaagaattgacagaacattacttatccaaaggatattcacgcaatgatcgtgttggaaaatcttacctagaatatcaatatgaagatgtattgcgtggtaagaagaaagaaatgaaatacacaacggacaaatctggaaaagttacatcttcagaagtgttaaatcctggcgctcgcggtcaagatttgaaattaacaatcgatatagatcttcaaaaagaagtagaatcattattagataaacaaattaagaagcttcgcagccaaggtgccaaagatatggataatgctatgatggttgtacaaaatcctaaaaatggagacattcttgcgcttgccggaaagcagattaataagagtggtaaaatgactgattatgacattggtacgtttacttctcaatttgcggttggatcttctgtaaaaggtggaacattattagctggttatcagaataaagctatcaaagttggagaaacaatggtcgatgaaccattacatttccaaggtggtttgacaaaacgatcatactttaataaaaacgggcatgtatctattaatgataagcaagctttgatgcattcatcaaacgtatatatgtttaaaacagcattaaaattagcgggagacccttattattctggtatggctttaccttcagacataagttcacctgcccaaaagctaagaagaggattaaatcaagtaggtttaggtgtgaaaacaggaatagatttaccaaatgaaacaagaggtcaaatcgaaccattaacaaataatccaggtaattatctagatttatcaattggtcaatatgatacctatacaccattacaattatcacaatatgtttcaactatagcgaatgatggttatagaatacagccacacattggattaacgattcatgaatcaactaataaagatgaggttggtccactcaagaagaaaattaatggcaccgtattgaacaaggttaataatactgaaaaagaaatcaaacaaattcaagaaggattcaaaatggcatttaatgataaagatggtactggatatgttagttttaaagatacagtagtccctactgctggtaaaacgggtaccgctgaagtgttccaaaacggagagccaagagttaactctacttatataggatacgcgccaattgatgatccaaaattagcgttttcaattgtatatacaaatcagcctgtaccaccaccatggttaacaggtggagacttaggtagagatgtaattaactactactttaagcagttaggtaaagatgataaaaataaagacaaagacaaataa

SEQ ID NO: 120 - DNA, 1071 bp, Staphylococcus aureus
atgacaaaagaaaatatttgtatcgtttttggagggaaaagtgcagaacacgaagtatcgattctgacagcacaaaatgtattaaatgcaatagataaagacaaatatcatgttgatatcatttatattaccaatgatggtgattggagaaagcaaaataatattacagctgaaattaaatctactgatgagcttcatttagaaaatggagaggcgcttgagatttcacagctattgaaagaaagtagttcaggacaaccatacgatgcagtattcccattattacatggtcctaatggtgaagatggcacgattcaagggctttttgaagttttggatgtaccatatgtaggaaatggtgtattgtcagctgcaagttctatggacaaacttgtaatgaaacaattatttgaacatcgagggttaccacagttaccttatattagtttcttacgttctgaatatgaaaaatatgaacataacattttaaaattagtaaatgataaattaaattacccagtctttgttaaacctgctaacttagggtcaagtgtaggtatcagtaaatgtaataatgaagcggaacttaaagaaggtattaaagaagcattccaatttgaccgtaagcttgttatagaacaaggcgttaacgcacgtgaaattgaagtagcagttttaggaaatgactatcctgaagcgacatggccaggtgaagtcgtaaaagatgtcgcgttttacgattacaaatcaaaatataaagatggtaaggttcaattacaaattccagctgacttagacgaagatgttcaattaacgcttagaaatatggcattagaggcattcaaagcgacagattgttctggtttagtccgtgctgatttctttgtaacagaagacaaccaaatatatattaatgaaacaaatgcaatgcctggatttacggctttcagtatgtatccaaagttatgggaaaatatgggcttatcttatccagaattgattacaaaacttatcgagcttgctaaagaacgtcaccaggataaacagaaaaataaatacaaaattgactaa

SEQ ID NO: 121 - DNA, 801 bp, Staphylococcus aureus
atgaataaaccaataggtgtaatagactctggtgtcggaggtttgacagtagctaaagaaattatgcgtcagttgccaaatgagacgatttattacttaggtgatattggacgatgtccatatgggccaagaccaggagaacaagtaaaacaatatacagttgaaatcgctcgtaaattaatggaatttgatataaaaatgctcgtgattgcttgtaatacagcaactgctgtagctttagaatatttacaaaagaccttatcaatcccagtgattggtgtaattgaaccaggtgctagaacagcaataatgactactagaaatcaaaatgtattagtactaggaactgaaggcacaattaaatctgaagcatatcgtacgcatattaaacgtatcaatccacatgtagaggtacatggcgttgcctgtccaggttttgtgccacttgtagaacaaatgagatatagtgatccaacaattacaagcattgtcattcatcaaacactgaaacgttggcgtaatagtgagtctgatactgtcattttaggatgtacccactatccattgctctataaacctatctatgattattttggtggtaaaaagacagtgatttcgtctggattagaaacggctcgtgaagttagtgcattgctaacatttagtaatgaacatgcaagttatactgaacatccagatcatcgattttttgcaacaggtgatcctactcacattactaacattatcaaagagtggttaaatttatctgtcaatgtggaacgtatatcagtgaatgactag

SEQ ID NO: 122 - DNA, 2235 bp, Staphylococcus aureus
atggcgaagcaaaaaattaaaattaaaaaaaataaaataggggcagtcctacttgttggtttattcggactgctcttttttatattggttttaagaatttcatatatcatgattactggacattctaatggtcaagatttagtcatgaaggcaaatgaaaagtatttagttaagaatgcacaacaaccagaacgaggaaagatatatgatcgtaatggtaaagtgctagcagaagatgtagaaagatataaacttgttgcagtaatagataaaaaggcgagtgccaattctaaaaaacctaggcatgtagttgataaaaaagagactgcaaagaaattatctacagtcattaatatgaagccagaggaaattgaaaagagacttagtcaaaagaaagctttccaaattgaatttggacgcaaaggaacaaatttaacgtatcaggacaaattgaaaatagagaaaatgaatttgcctggtatttctttattgcctgaaacagaacgcttttatccaaatggcaattttgcatcacacttaattggtagagctcagaaaaatccggatactggtgaacttaaaggtgcacttggagttgaaaagatttttgatagttatttaagtggatctaaaggatcattgagatatattcatgatatttggggatatatcgcaccaaatactaaaaaagagaagcagcctaaacgtggtgatgatgtccatttaacaatcgattcaaatattcaagtatttgttgaagaagctttagatggcatggttgaaagataccagccgaaagatttatttgcggttgtcatggatgccaaaactggagaaattttagcatacagtcagcgaccaacatttaatcctgaaactggtaaagactttggtaaaaagtgggcaaatgacctttatcaaaacacatacgagcctggatcaacatttaaatcatatgggttagcagctgctattcaagaaggtgcttttgatcctgataagaaatataaatctggacatagagatattatgggttcacgtatttcagactggaatagagtcggttggggtgaaatcccaatgtcactcggatttacttattcatctaatacattgatgatgcatttacaagatttagttggtgcagacaaaatgaaatcttggtatgaacgatttggatttggaaaatcaactaaaggtatgtttgatggagaagcacctggtcaaattggatggagtaatgagttgcaacaaaaaacgtcatcatttggtcaatcgacaacagtaacacctgttcaaatgttacaagcgcaatcagcgttctttaatgatggtaatatgttaaaaccatggtttgtgaatagcgttgaaaatcctgttagtaaaagacaattttataaagggcaaaaacaaatcgcaggcaaaccaataacaaaagatactgctgaaaaagttgaaaagcaattggatttagttgtgaatagtaagaagagtcacgctgcaaactatcgtattgatggttatgaggtcgaaggtaagactggtacagcacaagtcgctgcacctaatggtggtggatacgttaaaggtccaaacccatattttgtaagttttatgggtgacgcgccgaagaaaaatcctaaagttattgtatacgctggtatgagcttggcacaaaaaaatgaccaagaagcttatgaattaggtgttagtaaagcgtttaaaccaataatggaaaatactttgaaatatttaaatgtaggtaaatcaaaagatgacacatctaatgcagagtatagtaaagtgccagatgttgaaggtcaagacaaacaaaaagctattgataatgtgagtgcaaaatcattagaaccagttactattggttctggcacacaaataaaagcacaatctataaaagcagggaataaagtcttacctcatagtaaagtactgttattaacagatggagacttaactatgcctgacatgtcaggatggacgaaagaagatgtcattgcttttgaaaacctaacaaatattaaagtaaatttaaaaggtagcggttttgtgtcccaccaatcaattagtaagggacaaaaacttactgaaaaagataaaatagacgtagaattttcatcagagaatgtagacagcaattcgacgaataattctgattcaaattcagatgataagaagaaatctgacagtaaaactgacaaggataagtcggactaa

SEQ ID NO: 123 - DNA, 966 bp, Staphylococcus aureus
atgatttttgtatatgcgttattagcgctagtgattacatttgttttggtacctgttttaatacctacattaaaaaggatgaaatttggtcaaagtattcgagaagaaggtccacaaagccatatgaagaagactggtacaccaacgatgggtggactaacatttctattaagtattgtgataacgtctttggtggctattatatttgtagatcaagctaatccaatcatactgttattatttgtgacgattggttttgggttaattggttttatagatgattatattattgttgttaaaaagaataaccaaggtttaacaagtaaacagaagtttttggcgcaaattggtattgcgattattttctttgttttaagtaatgtgtttcatttggtgaatttttctacgagcatacatattccatttacgaatgtagcaatcccactatcatttgcatatgttattttcattgttttttggcaagtaggtttttctaatgcggtaaatttaacagatggtttagatggattagcaactggactgtcaattatcggatttacaatgtatgccatcatgagctttgtgttaggagaaacggcaattggtattttctgtatcattatgttgtttgcacttttaggatttttaccatataacattaaccctgctaaagtgtttatgggagatacaggtagcttagctttaggtggtatatttgctacgatttcaatcatgcttaatcaggaattatcattaatttttataggtttagtattcgtaattgaaacattatctgttatgttacaagtcgctagctttaaattgactggaaagcgtatatttaaaatgagtccgattcatcatcattttgaattgataggatggagcgaatggaaagtagttacagtattttgggctgttggtctgatttcaggtttaatcggtttatggattggagtgcattaa

SEQ ID NO: 124 - DNA, 1314 bp, Staphylococcus aureus
atgagacagtggacggcaatccatctagcgaaattggcgcgtaaagcaagtagagcagtaggtaaaagaggaacagatttacctggacaaatcgctagaaaagtggatacagatatattaagaaaattagcagagcaagttgatgatattgtatttatcagtggaacaaatggtaaaacaacgacttcaaacttaattggacatactttaaaagcaaataatattcaaattatacacaataatgaaggtgctaatatggctgcaggtataacttttgcattcatcatgcaatcaacacctaagactaaaattgcggtaatcgaaattgatgaaggttcgattccacgtgtgttaaaagaagttacaccttcaatgatggtatttactaatttctttagagatcaaatggatcgcttcggtgaaattgatattatggttaataacattgcagagacaattagtaataaaggcatcaaattattgctaaatgctgatgatccatttgtgagtcgtttgaaaatcgcaagtgatacgattgtgtactatggtatgaaagcacatgcccatgaatttgaacaaagtacgatgaatgaaagtagatattgtccaaactgtggtcgcttattgcaatacgattatattcattataatcaaattggtcattatcactgtcagtgtggtttcaaacgagagcaagcaaaatatgaaatatcaagttttgatgtggcaccgtttttatatttaaatatcaatgatgaaaaatatgatatgaaaattgcaggtgactttaacgcttataacgcgttagcagcatatactgttttaagagagctagggttaaatgaacaaacaattaaaaatggctttgaaacgtatacatcagacaatggtcgtatgcagtactttaaaaaagaacgaaaagaagcgatgatcaatttagctaaaaatcctgcaggaatgaatgcaagtttatcagttggtgaacaattagaaggcgaaaaagtgtatgttatttcgctaaatgataacgctgcagatggtcgagatacttcatggatttatgatgcagattttgaaaaattatctaagcaacaaattgaagctatcatcgtgacaggtacacgagcagaagaacttcaattgcgattgaagttagcagaggttgaagtaccaattatagttgagcgtgatatttataaagcaacggcaaagactatggattataaaggtttcacagttgcaataccaaactatacatcattagcgcctatgcttgaacaattaaaccgttcgtttgaaggaggtcaatcataa

SEQ ID NO: 125 - DNA, 1266 bp, Staphylococcus aureus
atggaaaagatgcatatcactaatcaggaacatgacgcatttgttaaatcccacccaaatggagatttattacaattaacgaaatgggcagaaacaaagaaattaactggatggtacgcgcgaagaatcgctgtaggtcgtgacggtgaagttcagggtgttgcgcagttactttttaaaaaagtacctaaattaccttatacgctatgttatatttcgcgtggttttgttgttgattatagtaataaagaagcgttaaatgcattgttagacagtgcaaaagaaattgctaaagctgagaaagcgtatgcaattaaaatcgatcctgatgttgaagttgataaaggtacagatgctttgcaaaatttgaaagcgcttggttttaaacataaaggatttaaagaaggtttatcaaaagactacatccaaccacgtatgactatgattacaccaattgataaaaatgatgatgagttattaaatagttttgaacgccgaaatcgttcaaaagtgcgcttggctttaaagcgaggtacgacagtagaacgatctgatagagaaggtttaaaaacatttgctgagttaatgaaaatcactggggaacgcgatggcttcttaacgcgtgatattagttactttgaaaatatttatgatgcgttgcatgaagatggagatgctgaactatttttagtaaagttggatccaaaagaaaatatagcgaaagtaaatcaagaattgaatgaacttcatgccgaaattgctaaatggcagcagaagatgaaaacatctgaaaagcaagctaaaaaagcgcaaaatatgattaatgatgcgcaaaataaaattgctaaaaatgaagatttaaaacgagacctagaagctttagaaaaggaacatcctgaaggtatttatctttctggtgcactattaatgtttgctggctcaaaatcatattacttatatggtgcgtcttctaatgaatttagagattttttaccaaatcatcatatgcagtatacgatgatgaagtatgcacgtgaacatggtgcaacaacttacgatttcggtggtacagataatgatccagataaagactcagaacattatggattatgggcatttaaaaaagtgtggggaacatacttaagtgaaaagattggtgaatttgattatgtattgaatcagccattgtaccaattaattgagcaagttaaaccgcgtttaacaaaagctaaaattaaaatatctcgtaaattaaaacgaaaatag

SEQ ID NO: 126 - DNA, 1263 bp, Staphylococcus aureus
atgaagtttacaaatttaacagctaaagagtttggtgcctttacagatagcatgccatacagtcatttcacgcaaactgttggccactatgagttaaagcttgctgaaggttatgaaacacatttagtgggaataaaaaacaataataacgaggtcattgcagcttgcttacttactgctgtacctgttatgaaagtgttcaagtatttttattcaaatcgcggtccagtgattgattatgaaaatcaagaactcgtacactttttctttaatgaattatcaaaatatgttaaaaaacatcgttgtctatacctacatatcgatccatatttaccatatcaatacttgaatcatgatggcgagattacaggtaatgctggtaatgattggttctttgataaaatgagtaacttaggatttgaacatactggattccataaaggatttgatcctgtgctacaaattcgttatcactcagtgttagatttaaaagataaaacagcagatgacatcattaaaaatatggatggacttagaaaaagaaacacgaaaaaagttaaaaagaatggtgttaaagtaagatttttatctgaagaagaactaccaatttttagatcatttatggaagatacgtcagaatcaaaagcttttgctgatcgtgatgacaaattttactacaatcgcttaaaatattacaaagaccgtgtgttagtacctttagcgtatatcaactttgatgaatatattaaagaactaaacgaagagcgtgatattttaaataaagatttaaataaagcgttaaaggatattgaaaaacgtcctgaaaataaaaaagcacataacaagcgagataacttacaacaacaacttgatgcaaatgagcaaaagattgaagaaggtaaacgtctacaagaagaacatggtaatgaattacctatctctgctggtttcttctttatcaatccatttgaagttgtttattatgctggtggtacatcaaatgcattccgtcattttgccggaagttatgcagtgcaatgggaaatgattaattatgcattaaatcatggcattgaccgttataatttctatggtgttagtggtaaatttacagaagatgctgaagatgctggtgtagttaaattcaaaaaaggttacaatgctgaaattattgaatatgttggtgactttattaaaccaattaataaacctgtttacgcagcatataccgcacttaaaaaagttaaagacagaattttttag

SEQ ID NO: 127 - DNA, 1266 bp, Staphylococcus aureus
atggataaaatagtaatcaaaggtggaaataaattaacgggtgaagttaaagtagaaggtgctaaaaatgcagtattaccaatattgacagcatctttattagcttctgataaaccgagtaaattagttaatgttccagctttaagtgatgtagaaacaataaataatgtattaacaactttaaatgctgacgttacatacaaaaaggacgaaaatgctgttgtcgttgatgcaacaaagactctaaatgaagaggcaccatatgaatatgttagtaaaatgcgtgcaagtattttagttatgggacctcttttagcaagactaggacatgctattgttgcattgcctggtggttgtgcaattggaagtagaccgattgagcaacacattaaaggttttgaagctttaggcgcagaaattcatcttgaaaatggtaatatttatgctaatgctaaagatggattaaaaggtacatcaattcatttagattttccaagtgtaggagcaacacaaaatattattatggcagcatcattagctaagggtaagactttaattgaaaatgcagctaaagaacctgaaattgtcgatttagcaaactacattaatgaaatgggtggtagaattactggtgctggtacagacacaattacaatcaatggtgtagaatcattacatggtgtagaacatgctatcattccagatagaattgaagcaggcacattactaatcgctggtgctataacgcgtggtgatatttttgtacgtggtgcaatcaaagaacatatggcgagtttagtctataaactagaagaaatgggcgttgaattggactatcaagaagatggtattcgtgtacgtgctgaaggggaattacaacctgtagacatcaaaacactaccacatcctggattcccgactgatatgcaatcacaaatgatggcattgttattaacggcaaatggtcataaagtcgtaaccgaaactgtttttgaaaaccgttttatgcatgttgcagagttcaaacgtatgaatgctaatatcaatgtagaaggtcgtagtgctaaacttgaaggtaaaagtcaattgcaaggtgcacaagttaaagcgactgatttaagagcagcagcagccttaattttagctggattagttgctgatggtaaaacaagcgttactgaattaacgcacctagatagaggctatgttgacttacacggtaaattgaagcaattaggtgcagacattgaacgtattaacgattaa

SEQ ID NO: 128 - DNA, 1071 bp, Staphylococcus aureus
atgacgaaaatcgcatttaccggagggggaacagttggacacgtatcagtaaatttaagtttaattccaactgcattatcacaaggttatgaagcgctttatattggttctaaaaatggtattgaaagagaaatgattgaatcacaactaccagaaattaagtattatcctatttcgagtggtaaattaagaagatatatttctttagaaaatgccaaagacgtatttaaagtattgaaaggtattcttgatgctcgtaaagttttgaaaaaagaaaaacctgatctattattttcaaaaggtggatttgtatctgtgcctgttgttattgcagccaaatcattaaatataccaactattattcatgaatctgacttaacaccaggattagcgaataagatagcacttaaatttgccaagaaaatatatacaacatttgaagaaacgctaaactacttacctaaagagaaagctgattttattggagcaacaattcgagaagatttaaaaaatggtaatgcacataatggttatcaattaacaggctttaatgaaaataaaaaagttttacttgttatgggtggaagcttaggaagtaaaaaattaaatagcattattcgcgaaaacttagatgcattattacaacaatatcaagtgatacatttaactggtaaaggattaaaagatgctcaagttaaaaaatcaggatatatacaatatgaatttgttaaagaggatttaacagatttattagcaattacggatacagtaataagtagagctggatcaaatgcgatttatgagttcttaacattacgtataccaatgttattagtaccattaggtttagatcaatcccgaggcgaccaaattgacaatgcaaatcattttgctgataaaggttatgctaaaacgattgatgaagaacaattaacagcacaaattttattacaagaactaaatgaaatggaacaggaaagaactcgaattatcaataatatgaaatcgtatgaacaaagttatacgaaagaagctttatttgataagatgattaaagacgcattgaattaa

SEQ ID NO: 129 - DNA, 1314 bp, Staphylococcus aureus
atgacacactatcattttgtcggaattaaaggttctggcatgagttcattagcacaaatcatgcatgatttaggacatgaagttcaaggatcggatattgagaactacgtatttacagaagttgctcttagaaataaggggataaaaatattaccatttgatgctaataacataaaagaagatatggtagttatacaaggtaatgcattcgcgagtagccatgaagaaatagtacgtgcacatcaattgaaattagatgttgtaagttataatgattttttaggacagattattgatcaatatacttcagtagctgtaactggtgcacatggtaaaacttctacaacaggtttattatcacatgttatgaatggtgataaaaagacttcatttttaattggtgatggcacaggtatgggattgcctgaaagtgattatttcgcttttgaggcatgtgaatatagacgtcactttttaagttataaacctgattacgcaattatgacaaatattgatttcgatcatcctgattattttaaagatattaatgatgtttttgatgcattccaagaaatggcacataatgttaaaaaaggtattattgcttggggtgatgatgaacatctacgtaaaattgaagcagatgttccaatttattattatggatttaaagattcggatgacatttatgctcaaaatattcaaattacggataaaggtactgcttttgatgtgtatgtggatggtgagttttatgatcacttcctgtctccacaatatggtgaccatacagttttaaatgcattagctgtaattgcgattagttatttagagaagctagatgttacaaatattaaagaagcattagaaacgtttggtggtgttaaacgtcgtttcaatgaaactacaattgcaaatcaagttattgtagatgattatgcacaccatccaagagaaattagtgctacaattgaaacagcacgaaagaaatatccacataaagaagttgttgcagtatttcaaccacacactttctctagaacacaagcatttttaaatgaatttgcagaaagtttaagtaaagcagatcgtgtattcttatgtgaaatttttggatcaattagagaaaatactggcgcattaacgatacaagatttaattgataaaattgaaggtgcatcgttaattaatgaagattctattaatgtattagaacaatttgataatgctgttgttttatttatgggtgcaggtgatattcaaaaattacaaaatgcatatttagataaattaggcatgaaaaatgcgttttaa

SEQ ID NO: 130 - DNA, 1482 bp, Staphylococcus aureus
ttggatgcaagtacgttgtttaagaaagtaaaagtaaagcgtgtattgggttctttagaacaacaaatagatgatatcactactgattcacgtacagcgagagaaggtagcatttttgtcgcttcagttggatatactgtagacagtcataagttctgtcaaaatgtagctgatcaagggtgtaagttggtagtggtcaataaagaacaatcattaccagctaacgtaacacaagtggttgtgccggacacattaagagtagctagtattctagcacacacattatatgattatccgagtcatcagttagtgacatttggtgtaacgggtacaaatggtaaaacttctattgcgacgatgattcatttaattcaaagaaagttacaaaaaaatagtgcatatttaggaactaatggtttccaaattaatgaaacaaagacaaaaggtgcaaatacgacaccagaaacagtttctttaactaagaaaattaaagaagcagttgatgcaggcgctgaatctatgacattagaagtatcaagccatggcttagtattaggacgactgcgaggcgttgaatttgacgttgcaatattttcaaatttaacacaagaccatttagattttcatggcacaatggaagcatacggacacgcgaagtctttattgtttagtcaattaggtgaagatttgtcgaaagaaaagtatgtcgtgttaaacaatgacgattcattttctgagtatttaagaacagtgacgccttatgaagtatttagttatggaattgatgaggaagcccaatttatggctaaaaatattcaagaatctttacaaggtgtcagctttgattttgtaacgccttttggaacttacccagtaaaatcgccttatgttggtaagtttaatatttctaatattatggcggcaatgattgcggtgtggagtaaaggtacatctttagaaacgattattaaagctgttgaaaatttagaacctgttgaagggcgattagaagttttagatccttcgttacctattgatttaattatcgattatgcacatacagctgatggtatgaacaaattaatcgatgcagtacagccttttgtaaagcaaaagttgatatttttagttggtatggcaggcgaacgtgatttaactaaaacgcctgaaatggggcgagttgcctgtcgtgcagattatgtcattttcacaccggataatccggcaaatgatgacccgaaaatgttaacggcagaattagccaaaggtgcaacacatcaaaactatattgaatttgatgatcgtgcagaagggataaaacatgcaattgacatagctgagcctggggatactgtcgttttagcatcaaaaggaagagaaccatatcaaatcatgccagggcatattaaggtgccacatcgagatgatttaattggccttgaagcagcttacaaaaagttcggtggtggccctgttgattaa

SEQ ID NO: 131 - DNA, 771 bp, Staphylococcus aureus
atgtttaaaaagctaataaataaaaagaacactataaataattataatgaagaattagactcgtctaatatacctgaacatatcgctattattatggatggtaatgggcgatgggctaagaagcgaaaaatgcctagaattaaaggtcattacgaaggtatgcaaacaataaaaaaaattactagggtagctagtgatattggtgttaagtacttaactttatacgccttttccactgaaaattggtcaagacctgaaagtgaagtaaattatattatgaatttgcctgtcaatttcttaaagacattcttaccggaactaattgaaaaaaatgtcaaagttgaaacaattggatttactgataagttgccaaaatcaacgatagaagcaattaataatgctaaagaaaagacagctaataataccggcttaaaattaatatttgcaattaattatggtggcagagcagaacttgttcatagtattaaaaatatgtttgacgagcttcatcaacaaggtttaaatagtgatatcatagatgaaacatatataaacaatcatttaatgacaaaagactatcctgatccagagttgttaattcgtacttcaggagaacaaagaataagtaatttcttgatttggcaagtttcgtatagtgaatttatctttaatcaaaaattatggcctgactttgacgaagatgaattaattaaatgtataaaaatttatcagtcacgtcaaagacgctttggcggattgagtgaggagtag